(19) United States
(12) Patent Application Publication
(10) Pub. No.: US 2004/0063120 A1
(43) Pub. Date: Apr. 1, 2004

(54) Expression profile of lung cancer

(75) Inventors: David G. Beer, Chelsea, MI (US); Sharon Kardia, Ann Arbor, MI (US); Thomas J. Giordano, Ann Arbor, MI (US); Jeremy Taylor, Ann Arbor, MI (US); Samir M. Hanash, Ann Arbor, MI (US); Chiang-Ching Huang, Naperville, IL (US); David E. Misek, Ann Arbor, MI (US); Dafydd Thomas, Fenton, MI (US); Rork Kuick, Dexter, MI (US)

Correspondence Address: David A. Casimir, MEDLEN & CARROLL, LLP, Suite 350, 101 Howard Street, San Francisco, CA, 94105, US

(73) Assignee: The Regents of the University of Michigan, Ann Arbor, MI

(21) Appl. No.: 10/617,067

(22) Filed: Jul. 10, 2003

Related U.S. Application Data
(60) Provisional application No. 60/394,858, filed on Jul. 10, 2002.

(51) Int. Cl. C12Q 1/68; G01N 33/574

(52) U.S. Cl. 435/6; 435/7.23

(57) ABSTRACT

The present invention relates to compositions and methods for cancer diagnostics, including but not limited to, cancer markers. In particular, the present invention provides gene expression profiles associated with lung cancers. Genes identified as cancer markers find use in the diagnosis and characterization of lung cancer. In addition, the genes provide targets for cancer drug screens and therapeutic applications.

The present application claims priority to U.S. Provisional Patent Application Serial No. 60/394,858, filed on Jul. 10, 2002, the disclosure of which is hereby incorporated by reference in its entirety.

This invention was made with government support under National Cancer Institute grant U19 CA-84953. The Government has certain rights in the invention.

### FIELD OF THE INVENTION

The present invention relates to compositions and methods for cancer diagnostics, including but not limited to, cancer markers. In particular, the present invention provides gene expression profiles associated with lung cancers. The present invention further provides novel markers useful for the diagnosis, characterization, and treatment of lung cancers.

### BACKGROUND OF THE INVENTION

Lung cancer remains the leading cause of cancer death in industrialized countries. About 75 percent of lung cancer cases are categorized as non-small cell lung cancer (e.g., adenocarcinomas), and the other 25 percent are small cell lung cancer. Lung cancers are characterized in to several stages, based on the spread of the disease. In stage I cancer, the tumor is only in the lung and surrounded by normal tissue. In stage II cancer, cancer has spread to nearby lymph nodes. In stage III, cancer has spread to the chest wall or diaphragm near the lung, or to the lymph nodes in the mediastinum (the area that separates the two lungs), or to the lymph nodes on the other side of the chest or in the neck. This stage is divided into IIIA, which can usually be operated on, and stage IIIB, which usually cannot withstand surgery. In stage IV, the cancer has spread to other parts of the body.

Most patients with non-small cell lung cancer (NSCLC) present with advanced stage disease, and despite recent advances in multi-modality therapy, the overall ten-year survival rate remains dismal at 8-10% (Fry et al., Cancer 86:1867 [1999]). However, a significant minority of patients, approximately 25-30%, with NSCLC have pathological stage I disease and are usually treated with surgery alone. While it is known that 35-50% of patients with stage I disease will relapse within five years (Williams et al., Thorac. Cardiovasc. Surg. 82:70 [1981]; Pairolero et al., Ann, Thorac. Surg. 38:331 [1984]), it is not currently possible to identify which specific patients are at high risk of relapse. Adenocarcinoma is currently the predominant histologic subtype of NSCLC (Fry et al., supra; Kaisermann et al., Brazil Oncol. Rep. 8:189 [2001]; Roggli et al., Hum. Pathol. 16:569 [1985]). While histopathological assessment of primary lung carcinomas can roughly stratify patients, there is still an urgent need to identify those patients who are at high risk for recurrent or metastatic disease by other means. Previous studies have identified a number of preoperative variables that impact survival of patients with NSCLC (Gail et al., Cancer 54:1802 1984]; Takise et al., Cancer 61:2083 [1988]; Ichinose et al., J. Thorac. Cardiovasc. Surg. 106:90 [1993]; Harpole et al., Cancer Res. 55:1995]). Tumor size, vascular invasion, poor differentiation, high tumor proliferate index, and several genetic alterations, including K-ras (Rodenhuis et al., N. Engl. J. Med. 317:929 [1987]; Slebos et al., N. Engl. J. Med. 323:561 [1990]) and p53 (Harpole et al., supra; Horio et al., Cancer Res. 53:1 [1993]) mutation, have been reported as prognostic indicators.

Tumor stage is an important predictor of patient survival, however, much variability in outcome is not accounted for by stage alone, as is observed for stage I lung adenocarcinoma which has a 65-70% five-year survival (Williams et al., supra; Pairolero et al., supra). Current therapy for patients with stage I disease usually consists of surgical resection and no additional treatment (Williams et al., supra; Pairolero et al., supra). The identification of a high-risk group among patients with stage I disease would lead to consideration of additional therapeutic intervention for this group, as well as leading to improved survival of these patients.

### SUMMARY OF THE INVENTION

The present invention relates to compositions and methods for cancer diagnostics, including but not limited to, cancer markers. In particular, the present invention provides gene expression profiles associated with lung cancers.

Accordingly, in some embodiments, the present invention provides a method for characterizing lung tissue in a subject, comprising providing a lung tissue sample; and detecting a decreased or increased expression relative to a non-cancerous lung tissue control of a marker selected from the group consisting of AOE372, ATP5D, B4GALT, Ppase, GRP58, GSTM4, P4HB, TPI, and UCHL1, thereby characterizing the lung tissue sample. In some embodiments, the detecting comprises detecting three or more, and preferably 5 or more of the markers. In some embodiments, the expression is increased at least 1.4 fold, preferably at least 4 fold, and even more preferably at least 10 fold relative to the non-cancerous lung tissue control.

In some embodiments, the detecting the presence of expression of the marker comprises detecting the presence of mRNA corresponding to the marker. In some embodiments, detecting the presence of expression of the mRNA comprises exposing the mRNA to a nucleic acid probe complementary to the mRNA. In other embodiments, detecting the presence of expression of the marker comprises detecting the presence of a polypeptide corresponding to the marker. In some embodiments, detecting the presence of the polypeptide comprises exposing the polypeptide to an antibody specific to the polypeptide and detecting the binding of the antibody to the polypeptide.

In some embodiments, the subject comprises a human subject. In certain embodiments, the sample comprises biopsy tissue. In other embodiments, the sample comprises a lung cancer sample. In some embodiments, characterizing the lung tissue comprises identifying a stage of lung cancer in the lung tissue. In some embodiments, the stage is selected from the group including, but not limited to, stage I lung cancer, stage II lung cancer, and stage III lung cancer. In some embodiments, the method further comprises the step of providing a prognosis to the subject. In some embodiments, the prognosis comprises a risk of developing stage III lung cancer. In other embodiments, the prognosis comprises a survival chance.

The present invention further provides a method of characterizing lung cancer, comprising providing a lung cancer tissue sample; and detecting the level of expression relative to a non-cancerous lung tissue control of a marker selected from the group including, but not limited to, GRP-58, PSMC, VIM, SOD, TPI, thereby characterizing the lung cancer tissue sample. In some embodiments, an increased level of GRP-58, PSMC, and VIM is indicative of stage III lung cancer. In other embodiments, a decreased level of SOD and TPI is indicative of stage III lung cancer.

In some embodiments, the detecting the level of expression of the marker comprises detecting the amount of mRNA corresponding to the marker. In some embodiments, detecting the amount of expression of the mRNA comprises exposing the mRNA to a nucleic acid probe complementary to the mRNA. In other embodiments, detecting the presence of level of the marker comprises detecting the amount of a polypeptide corresponding to the marker. In some embodiments, detecting the amount of the polypeptide comprises exposing the polypeptide to an antibody specific to the polypeptide and detecting the binding of the antibody to the polypeptide. In some embodiments, the subject comprises a human subject. In certain embodiments, the sample comprises biopsy tissue. In other embodiments, the sample comprises a lung cancer sample.

The present invention further provides a method of predicting survival in lung cancer patients, comprising providing lung cancer tissue sample from a subject; detecting the presence or absence of a marker selected from the group including, but not limited to, CK19, CK7, and CK8 in the lung cancer tissue sample. In some embodiments, the presence of the marker is indicative of decreased survival of the subject.

In some embodiments, the detecting the presence of expression of the marker comprises detecting the presence of mRNA corresponding to the marker. In some embodiments, detecting the presence of expression of the mRNA comprises exposing the mRNA to a nucleic acid probe complementary to the mRNA. In other embodiments, detecting the presence of expression of the marker comprises detecting the presence of a polypeptide corresponding to the marker. In some embodiments, detecting the presence of the polypeptide comprises exposing the polypeptide to an antibody specific to the polypeptide and detecting the binding of the antibody to the polypeptide. In some embodiments, the subject comprises a human subject.

The present invention additionally provides a kit for characterizing lung cancer in a subject, comprising a reagent capable of specifically detecting the presence of absence of expression of a marker selected from the group including, but not limited to, of GRP58, PSMC, VIM, SOD, TPI, AOE372, ATP5D, B4GALT, Ppase, GRP58, GSTM4, P4HB, TPI, and UCHL1; and instructions for using the kit for characterizing cancer in the subject. In some embodiments, the reagent comprises a nucleic acid probe complementary to an mRNA corresponding to the marker. In other embodiments, the reagent comprises an antibody that specifically binds to a polypeptide corresponding to the marker. In some embodiments, the instructions comprise instructions required by the United States Food and Drug Administration for use in in vitro diagnostic products.

In still further embodiments, the present invention provides a kit for predicting survival in a lung cancer subject, comprising a reagent capable of specifically detecting the presence or absence of a marker selected from the group including, but not limited to, CK19, CK7, and CK8 in a lung cancer tissue sample; and instructions for using the kit for predicting survival in the subject. In some embodiments, the reagent comprises a nucleic acid probe complementary to an mRNA corresponding to the marker. In other embodiments, the reagent comprises an antibody that specifically binds to a polypeptide corresponding to the marker. In some embodiments, the instructions comprise instructions required by the United States Food and Drug Administration for use in in vitro diagnostic products.

In yet other embodiments, the present invention provides a method of screening compounds, comprising providing a lung cell sample; and one or more test compounds; and contacting the lung cell sample with the test compound; and detecting a change in expression of a marker selected from the group including, but not limited to, GRP-58, PSMC, VIM, SOD, TPI, AOE372, ATP5D, B4GALT, Ppase, GRP58, GSTM4, P4HB, TPI, UCHL1, CK19, CK7, and CK8 in the lung cell sample in the presence of the test compound relative to the absence of the test compound. In some embodiments, the detecting comprises detecting mRNA corresponding to the marker. In other embodiments, the detecting comprises detecting a polypeptide corresponding to the marker. In some embodiments, the cell is in vitro. In other embodiments, the cell is in vivo. In certain embodiments, the test compound comprises an antisense compound. In some embodiment, the test compound comprises a drug.

In still other embodiments, the present invention provides a lung cancer expression profile map comprising gene expression level information for two or more markers selected from the group including, but not limited to, BAG1, CASP4, FADD, P63, 5T4, ITGA2, KRT18, KRT19, KRT7, LAMB1, TMSB4X, TUBA1, BMP2, CDC6, H2AFZ, PDAP1, POLD3, REG1A, S100P, SERPINE1, STX1A, ADM, AKAP12, ARHE, DEFB1, GRB7, INHA, ITK, NACA, STC1, TNFAIP6, VEGF, VLDLR, WNT1, WNT10B, HSPA8, ERBB2, FXYD3, HLA-B, HPCAL1, P2RX5, PEX7, SLC20A1, SLC2A1, VDAC2, ALDH8, ALDOA, ATP2B1, CDS1, CSTB, CTSL, CYP24, FUCA1, FUT3, GAPD, GCNT1, HMBS, KYNU, MLN64, MSH3, MT2A, NME2, NP, PACE, PDE7A, PLGL, PPIF, PTPRCAP, RPC, SC4MOL, SLC1A6, UBC, UGP2, UQCRC2, COPEB, CRK, DBP, GARS, HRB, HSU53209, PRDM2, RELA, RPS26, RPS3, RPS6 KB1, SUI1, TIEG, TMF1, B1, FEZ2, HPIP, KIAA0005, KIAA0020, KIAA0084, KIAA0153, KIAA0263, KIAA0317 and MGB1. In some embodiments, the map is digital information stored in computer memory. In some embodiments, the map comprises information for three or more, preferably five or more, and even more preferably ten or more of the markers.

### DESCRIPTION OF THE FIGURES

FIG. 1 shows unsupervised classification analysis of lung adenocarcinomas.

FIG. 2 shows validation analyses of gene expression profiling. FIG. 2a shows Northern blot analysis of selected candidate genes for verification of data obtained from oligonucleotide arrays. FIG. 2b shows correlation analysis of quantitative data obtained from oligonucleotide arrays and Northern blots for the IGFBP3 and LDH-A genes.

FIG. 3 shows gene expression profiles and patient survival. FIG. 3a shows the relationship between tumor stage and patient survival. FIG. 3b shows the relationship between the observed survival in the 43 test samples and their risk assignments based on the 50-gene risk index estimated in the 43 training samples. FIG. 3c shows the relationship between patient survival and the risk assignments in test samples (FIG. 4B) conditional for tumor stage. FIG. 3d shows the relationship between observed survival in the test cases and their risk assignments based on the 86 leave-one out cross-validation of the 50 gene risk index. FIG. 3e shows the relationship between test case's risk assignment and survival (FIG. 4D) conditional on tumor stage. FIG. 3f shows the relationship between tumor class identified by hierarchical clustering and patient survival.

FIG. 4a shows gene expression patterns determined using agglomerative hierarchical clustering of the 86 lung adenocarcinoma against the 100 survival-related genes (Table 2) identified by the testing-training and cross-validation analysis. FIG. 4b shows that an outlier gene expression pattern (greater than five times the interquartile range among all samples) is observed for the erbB2 and Reg1A genes (top two panels). The S100P and crk genes (bottom panels) show a graded pattern of expression related to patient survival. FIG. 4c shows the number of outliers per person identified in the top 100 genes plotted by survival distribution.

FIG. 5 shows that transmembrane erbB2 protein expression is substantially increased in tumor L94 containing the amplified erbB2 gene.

FIG. 6 shows 2D-PAGE gel separation of proteins identified with silver staining from a stage I lung adenocarcinoma. FIG. 6A shows the entire gel. FIGS. 6B-F show the outlined areas of FIG. 6A showing proteins significantly increased in lung adenocarcinoma.

FIG. 7 shows protein expression frequencies in lung adenocarcinomas. The full name of each protein is shown in Table 3.

FIG. 8 shows 2D Western blot analysis of UCHL1 (A) and GRP58 (B) proteins.

FIG. 9A shows a digital image of a silver-stained 2D-PAGE separation of a stage I lung adenocarcinoma showing protein spots separated by molecular weight (MW) and isoelectric point (PI). FIG. 9B shows the outlined areas of (A) showing protein GRP58. FIG. 9C shows a 2D Western blot of GRP58 from the A549 lung adenocarcinoma cell line. FIG. 9D shows the outlined areas of (A) showing the protein isoforms of Op18 E, 2D Western blot of Op18 from A549 cells.

FIGS. 10A-C shows plots showing the correlation between mRNA and protein for the three selected genes: Op18, Annexin IV and GAPD for all 76 lung adenocarcinomas and 9 non-neoplastic lung samples (p<0.05). FIG. 10D shows a distribution of all 165 Spearman correlation coefficients (r) and verification analysis using SAM.

FIG. 11 shows the overall correlation of mRNA and protein levels across all 165 protein spots (3A) and across 28 protein spots which contained individual r values larger than 0.244 (3B).

FIG. 12 shows a 2D PAGE gel image of a stage I lung adenocarcinoma with proteins separated by molecular weight and isoelectric point. FIG. 12A shows the location of CK8 spots. FIG. 12B shows the location of CK7 spots. FIG. 12C shows the location of CK18 spots. FIG. 12D shows the location of CK19 spots.

FIG. 13A shows a 2D gel digital image of the silver-stained region shown in FIG. 12, Box A containing eight CK8 spots expressed in a primary lung adenocarcinoma. FIG. 13B shows a 2D Western blot analysis using anti-CK8 monoclonal antibody clone TS1 showing the eight isoforms quantified of the approximately twenty that are immunoreactive in A549 cells. FIG. 13C shows a 2D Western blot analysis using anti-CK7 monoclonal antibody clone OV-TL 12/30. The region is shown in FIG. 12, Box A. FIG. 13D shows a 2D Western blot analysis using anti-CK7 monoclonal antibody clone K72.7. FIG. 13E shows a 2D Western blot analysis using anti-CK18 monoclonal antibody clone DC-10. The region is shown in FIG. 12, Box C. FIG. 13F shows a 2D Western blot analysis using anti-CK19 monoclonal antibody clone M0772. The region is shown in FIG. 12, Box D.

### GENERAL DESCRIPTION

Adenocarcinomas constitute a biologically heterogeneous group of lung tumors, and are now the most common type of lung cancer. Although many insights into the molecular pathology of lung tumors have been achieved, additional information is critical to both the understanding of the development and progression of these tumors as well as to aid in early diagnosis. The analysis of genes overexpressed in lung cancer, and that they may serve as tumor markers, has been the subject of extensive research. The most commonly evaluated markers include neuron-specific enolase (NSE), carcinoembryonic antigen (CEA), cytokeratin 19 fragments (CYFRA 21-1), squamous cell carcinoma antigen (SCC), cancer antigen CA 125 (CA 125) and tissue polypeptide antigen (TPA) (Stieber et al., Anticancer Research, 19:2817 [1999]). Although the analysis of multiple biological markers may be more informative than the use of a single marker (Schneider et al., Br. J. Cancer, 83:473 [2000]), very few markers have been accepted for routine clinical use either due to conflicting reports, or because associations are insufficient for formulating clinical treatment plans (Alaiya et al., Electrophoresis 21:1210 [2000]). The detection of new candidate markers is complex due to the known heterogeneity of lung cancers.

Comprehensive gene expression profiling serves to define clinically relevant subsets of tumors not discernable by traditional approaches. Experiments conducted during the course of development of the present invention used several approaches for the analysis of gene expression data related to clinicopathological variables and patient survival. One approach, hierarchical clustering of all samples with all 4966 genes, was used to examine similarities among lung adenocarcinomas in their patterns of gene expression. Experiments conducted during the course of the development of the present invention found three clusters that showed significant differences with respect to tumor stage and tumor differentiation. The present invention is not limited to a particular mechanism. Indeed, an understanding of the mechanism is not necessary to practice the present invention. Nonetheless, it is contemplated that tumors with similar histological features of differentiation demonstrate similarities in gene expression. This feature also partly underlies the observed statistical association of tumor stage and cluster, as many of the higher stage tumors, often poorly differentiated and previously associated with a reduced survival (Ichisoe et al., supra; Harpole et al., supra), were located in Cluster 3. Although this cluster contained the highest percentage of stage III tumors, it also contained a nearly equal mixture of stage I and stage III tumors and not all tumors were poorly differentiated. This indicates a subset of stage I lung adenocarcinomas share gene expression profiles with higher stage tumors. 10 of the 11 stage I tumors found in Cluster 3 were the high risk stage I tumors identified using the risk index in the leave-one-out cross validation.

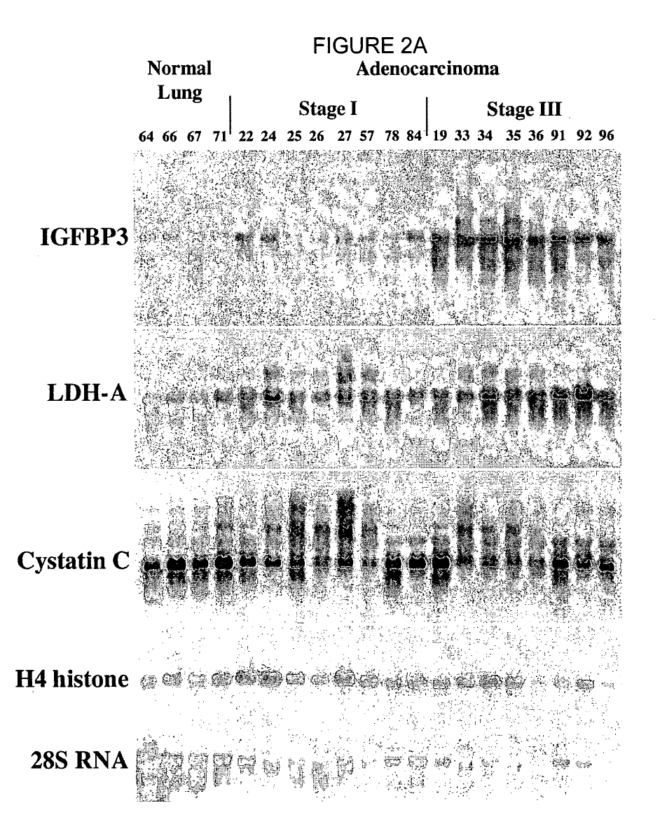

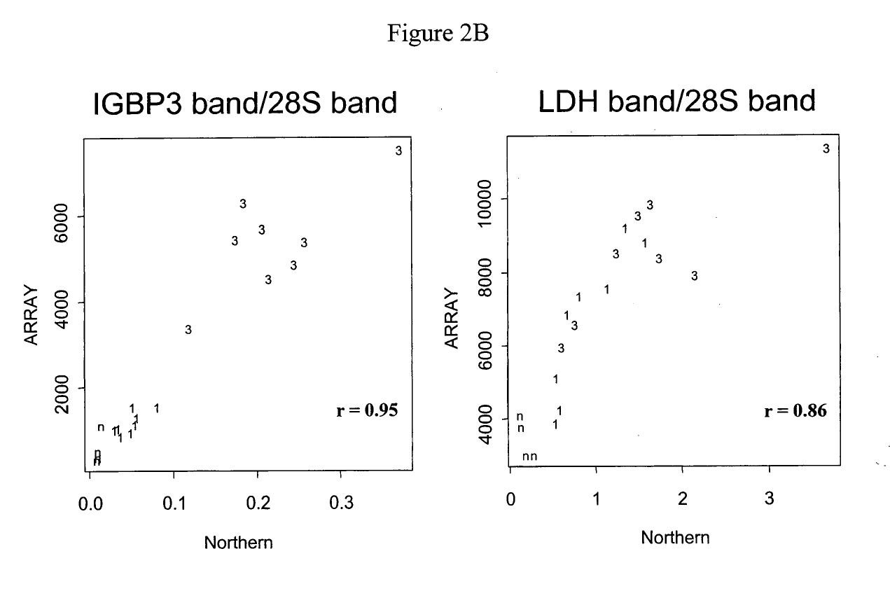

The present study utilized genes that differed significantly between specific comparison groups such as tumor stage to validate the expression data from the arrays. The strong correlation of northern blot hybridization and oligonucleotide array data for gene expression in the same samples (FIG. 2b) indicates that these studies provide robust gene expression estimates. Immunohistochemical analysis using the same tumor samples in tissue arrays further demonstrates protein expression within the lung tumor cells. These complementary expression analyses provide support that many of the genes identified using gene expression profiles are relevant to lung adenocarcinoma. For example, IGFBP3 gene expression is increased in lung adenocarcinomas (FIG. 2). This protein modulates the autocrine or paracrine effects of insulin-like growth factors and elevated IGFBP3 expression is observed in colon cancers (Kansra et al., Int. J. Cancer 87:373 [2000] and increased serum IGFBP3 is associated with progression in breast cancers (Vadgama et al., Oncology 57:330 [1999]).

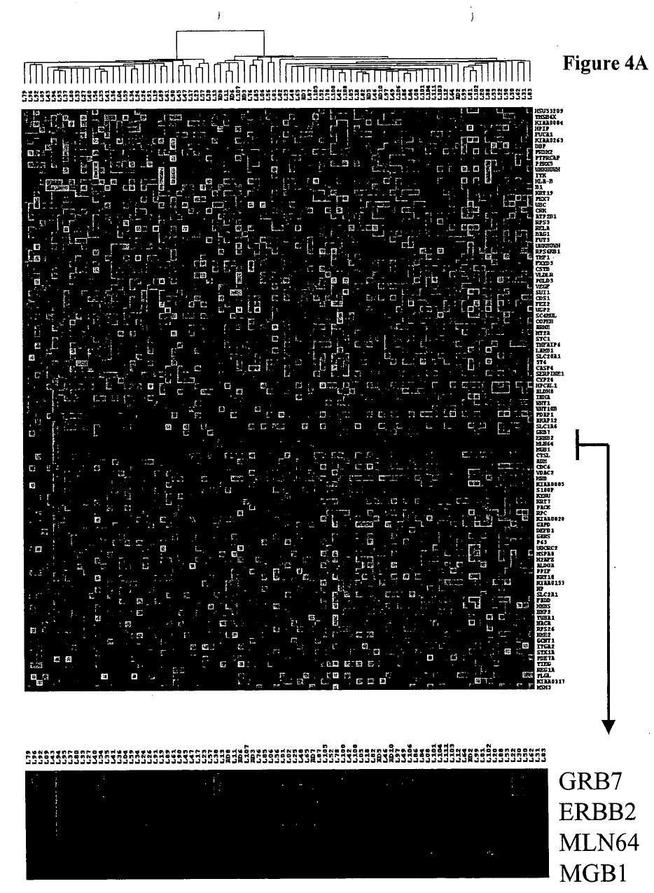

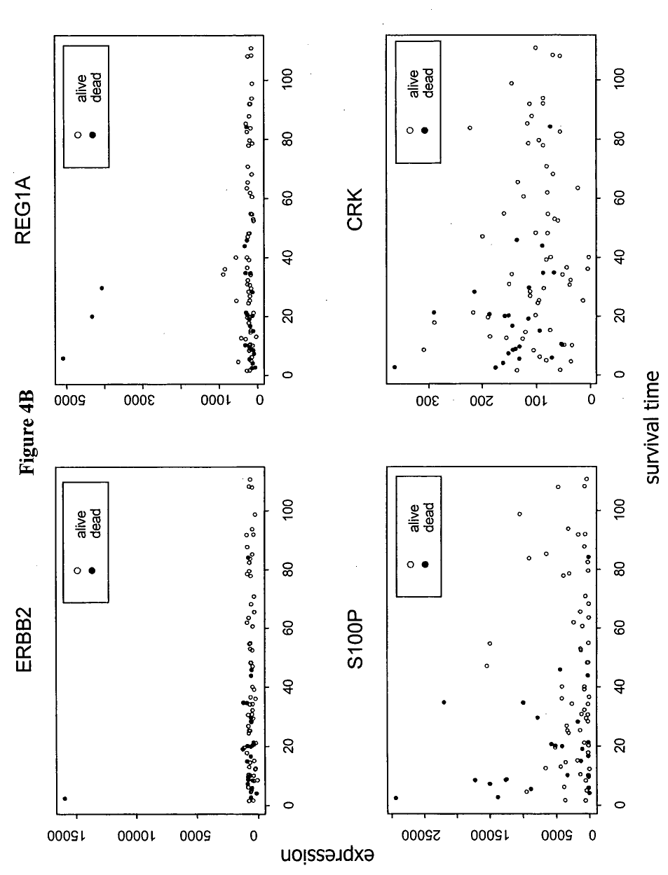

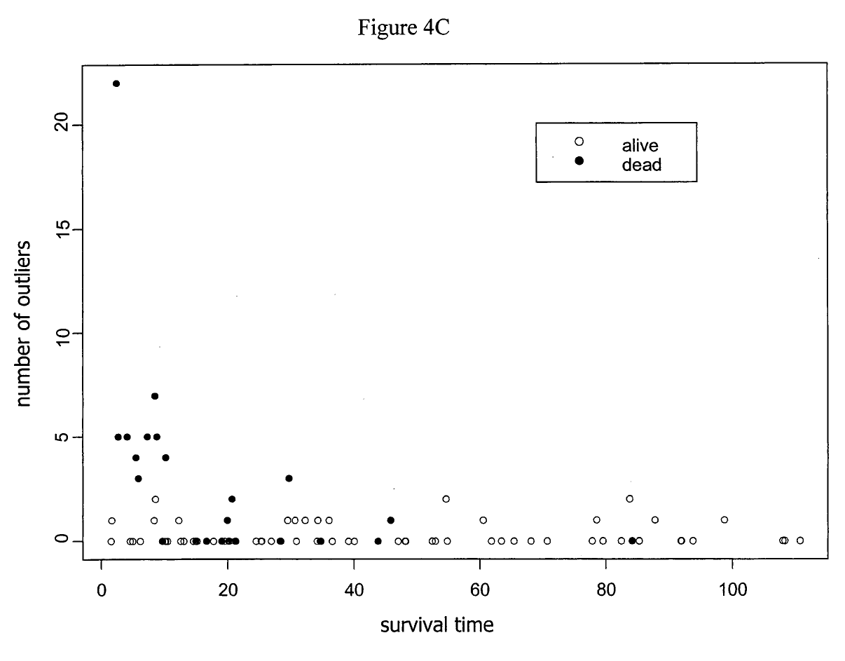

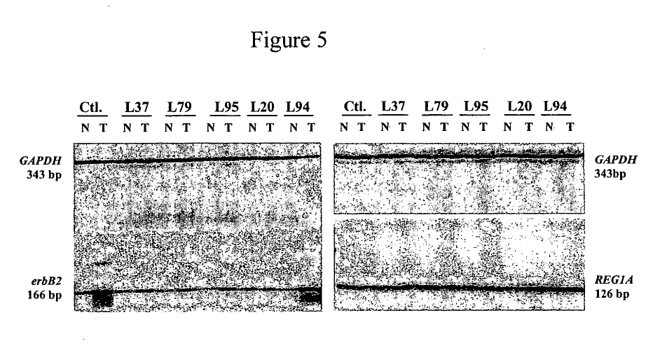

The cross validation analytical strategy used in the illustrative examples described herein is particularly informative for gene expression-disease outcome analyses (Golub et al., Science 286 531 [1999]; Hedenfalk N. Engl. J. Med. 344:539 [2001]) and identification of cross-validated genes with an even larger tumor cohort will help refine this risk index for use in a clinical setting. The gene expression data however, also provides opportunities to observe overarching patterns that advance the understanding of gene-disease associations. For example, the top 100 survival genes includes those involved in signaling, cell cycle and growth, transcription, translation, and metabolism. Expression of many of these genes is likely a function of increased proliferation and metabolism in the more aggressive tumors. Some genes, such as erbB2 and Reg1A (FIG. 4a,b), were highly overexpressed in a few patients having poor survival. In one tumor, the erbB2 gene was amplified (FIG. 5a), demonstrating that genomic changes may underlie the overexpression of a subset of these outlier genes. Immunohistochemistry confirmed protein overexpression in this patient's tumor (FIG. 5b). Seven of the eight outlier genes showed no evidence of gene amplification, indicating that other mechanisms underlie the increased mRNA expression of these survival-related genes.

Most genes showed a graded relationship between expression and patient survival. Genes such as vascular endothelial growth factor (VEGF), known to be strongly associated with survival in lung cancer (Ohta et al., Br. J. Cancer 76:1040 [1997]; Shibusa et al., Clin. Cancer Res. 4:1483 [1998]), demonstrated a graded expression pattern, as did the S100P and crk oncogene (FIG. 5b). S100P is a calcium-regulated protein not previously reported in lung cancer. The crk gene, the cellular homolog of the v-crk oncogene, is a member of a family of adaptor proteins involved in signal transduction and interacts directly with c-jun N-terminal kinase 1 (JNK1) (Girardin et al., EMBO J. 20:3437 [2001]. crk has not been previously reported in lung cancer. Genes were observed that were related to survival in many patients and other genes that related to survival in much smaller subsets of patients. This result is consistent with the complex molecular architecture of tumors in general, the heterogeneity of lung adenocarcinomas in particular and the multiple mechanisms underlying tumor cell survival and metastasis.

The results of experiments conducted during the course of development of the present invention demonstrate that a gene expression risk profile, based on the genes most associated with patient survival, can distinguish stage I lung adenocarcinomas with differing prognoses. It is evident that the particular genes that define the clusters, or are associated with survival, reflect the characteristics of the particular tumors included in the analysis. The genes associated with survival identified in this study serves to identify better markers for diagnosis or prognosis and new targets for therapeutic intervention.

Experiments conducted during the course of development further investigated protein expression in lung adenocarcinomas. Two-dimensional polyacrylamide gel electrophoresis (2D-PAGE) is able to simultaneously examine hundreds of polypeptides in a tissue sample. It has been widely used for the detection and identification of potential tumor markers (See e.g., Okuzawa et al., Electrophoresis, 15:382 [1994]). Experiments conducted during the course of development of the present invention analyzed 93 lung adenocarcinomas and 10 uninvolved lung samples for protein expression using 2D-PAGE. Analysis software was used to obtain quantitative measures for individual protein spots. Proteins of interest were identified using matrix-assisted laser desorption/ionization mass spectrometry (MALDI-MS) or peptide sequencing. Associations between the proteins that were overexpressed in the lung adenocarcinomas and clinical-pathological features of the tumor were determined. Evaluation of the same tumor samples for mRNA expression using oligonucleotide arrays was used to examine the mechanisms underlying the expression profiles identified by proteomic analysis.

A series of 93 lung adenocarcinomas (64 stage I and 29 stage III) and ten uninvolved lung samples were examined for quantitative differences in protein expression using two-dimensional polyacrylamide gel electrophoresis (2D-PAGE). Candidate proteins were identified using matrix-assisted laser desorption/ionization mass spectrometry or peptide sequencing. The levels of the individual isoforms of nine proteins found to be overexpressed in the lung tumors were examined. Potential mechanisms for overexpression were examined by comparing mRNA expression levels, assessed using oligonucleotide arrays, to the protein values in the same samples.

Previous studies have shown increased expression of specific enzyme proteins in lung cancer or elevated levels in the serum of lung cancer patients. These include neuron-specific enolase (NSE), lactate dehydrogenase, 5′-nucleotide phosphodiesterase, thymidine kinase, hexokinase, sialyltransferase, uridine kinase, glucose-6-phosphate dehydrogenase and ceruloplasmin (Wu, Annals of Clinical and Laboratory Science, 29:106 [1999]; Schwartz, Annals of Clinical and Laboratory Science, 7:99 [1977]). The increased expression of these proteins may reflect the overall changes in cellular metabolism and growth rate that occur during malignancy (Stefanini, Cancer, 55:1931 [1985]). The present invention provides proteins demonstrating increased expression in lung adenocarcinomas and changes in the expression of individual protein isoforms, reflecting post-translational patterns that are contemplated to correlate with their clinical-pathological features.

The candidate tumor markers in this study were found using quantitative assessment with 2D-PAGE gels and mass spectrometry and a number were increased in lung adenocarcinomas (1.4- to 10.6-fold) as compared to normal lung tissue. Among these, AOE372, PPase, GSTM4 and UCHL1 were increased 10.6-, 7.6-, 4.0- and 3.5-fold, respectively. The frequency of elevated expression in lung adenocarcinomas was found to range from 35.5% to 96.8% among the 93 tumors examined. GSTM4 was the most consistently overexpressed protein, being present in 96.8% of the tumors. Correlations were observed between overexpression of some proteins and specific clinical-pathological variables, including tumor differentiation (AOE372 and TPI), tumor sub-histology (PPase), a positive smoking history (PPase, TPI and UCHL1), a positive lymphocytic response (P4HB).

Both isoforms of the GRP58 were significantly correlated with their respective mRNA levels. GRP58 mRNA levels were also significantly increased in lung adenocarcinomas as compared to normal lung tissue. This suggests that the increase in protein expression in these tumors reflects increased GRP58 transcription. The lack of correlation observed among the other proteins with their mRNA levels may reflect other post-translational regulation differences.

In terms of biological processes, the proteins identified in this study can be divided into two main groups. One group includes the enzymes involved in energy-related pathways: ATP5D (energy generation), PPase (phosphate metabolism), TPI (glycolysis), B4GALT (lactose biosynthesis) and UCHL1 (protein degradation). The elevated expression of these enzymes may relate to the increased requirements of both energy and protein synthetic and degradation pathways in rapidly growing tumors. The other group of proteins may be involved in antioxidation or detoxification reactions or related pathways and include: AOE372 (oxidoreductase), P4HB (oxidoreductase), GRP58 (protein disulfide isomerase) and GSTM4 (stress response). These enzymes may help clear the toxic byproducts resulting from elevated metabolism in these tumors.

As a method of validation of the 2D-PAGE protein expression data, 2D Western blot and immunohistochemistry were utilized with the same antibodies. The specific isoforms identified by 2D Western blot of UCHL1 and GRP58, were revealed as the same spots identified by MS. Further, the expression of UCHL1 and GRP58 were substantially elevated within the cytoplasm of the tumor cells. UCHL1 (PGP9.5) belongs to the ubiquitin carboxyl-terminal hydrolase (UCH) family that is a part of the cellular proteolytic pathway that regulates many cellular processes, including cell cycle progression and cell death (Hibi et al., Am. J. Pathol., 155:711 [1999]).

PGP9.5 is widely expressed in neuronal tissues at all stages of differentiation. PGP9.5 mRNA and protein are highly expressed in lung cancer tissues (55%), independent of neuronal differentiation (Hibi et al., supra). Autoantibodies to PGP9.5 are detectable in the sera from 9 of 64 patients with lung cancer (Brichory et al., Cancer Res., 61:7908 [2001]). Some of the other proteins examined in experiments conduced during the course of development of the present invention have shown a prior relationship to cancer. GRP58, isoform of protein disulfide isomerase (PDI) is present in lung tissue (Koivunen et al., Genomics, 42:397 [1997]), and increased following the v-src transformation of normal rat kidney cells and NIH3T3 cells (Hirano et al., Eur. J. Biochem., 234:336 [1995]). GRP58 (isoform #353) expression was significantly increased in tumors containing the 12^{th}/13^{th} codon K-ras gene mutations. TPI, a key component of the glycolytic pathway that converts dihydroxyacetone phosphate to glyceraldehyde 3-phosphate, shows increased expression in bladder and colon carcinomas (Montgomerie et al., Clin. Biochem., 30:613 [1997]). Elevated expression of TPI mRNA was present in the lung tumors and TPI (isoform #1161) was significantly higher in stage m tumors relative to stage I tumors (P<0.02).

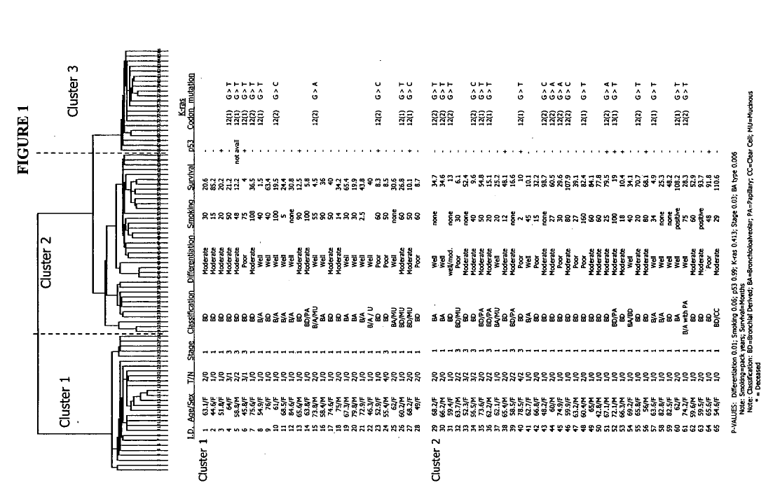

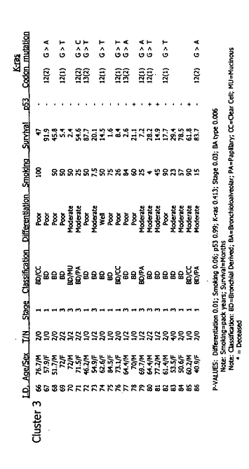

Because of differential protein processing and post-translational modifications, multiple protein isoforms of individual proteins can be identified on 2D gels. In experiments conducted during the course of development of the present invention, TPI, UCHL1, GRP58 and P4HB were shown to express two or three different isoforms respectively in lung tumors (FIG. 1). The present invention is not limited to a particular mechanism. Indeed, an understanding of the mechanism is not necessary to practice the present invention. Nonetheless, it is contemplated that these isoforms reflect phosphorylation or other modifications and were found to correlate with different clinical-pathological variables. For example, the levels of P4HB (isoform #320) were highly elevated in lung tumors but reduced in patients showing a positive lymphocytic response. These findings suggest that the different protein isoforms may be correlated with specific features or functions of lung tumors. Thus, they provide useful diagnostic information and further indicate the need to identify the specific modifications underlying these specific protein isoforms.

The relationship between gene expression measured at the mRNA level and the corresponding protein level is not well characterized in human cancer. Experiments conducted during the course of development of the present invention compared mRNA and protein expression for a cohort of genes in the same lung adenocarcinomas. The abundance of 165 protein spots representing 98 individual genes was analyzed in 76 lung adenocarcinomas and 9 non-neoplastic lung tissues using two-dimensional polyacrylamide gel electrophoresis. Specific polypeptides were identified using matrix-assisted laser desorption/ionization mass spectrometry. For the same 85 samples, mRNA levels were determined using oligonucleotide microarrays, allowing a comparative analysis of mRNA and protein expression among the 165 protein spots. Twenty-eight of the 165 protein spots (17%) or 21 of 98 genes (21.4%) had a statistically significant correlation between protein and mRNA expression (r>0.2445; p<0.05), however, among all 165 proteins the correlation coefficient values (r) ranged from −0.467 to 0.442. Correlation coefficient values were not related to protein abundance. Further, no significant correlation between mRNA and protein expression was found (r=−0.025) if the average levels of mRNA or protein among all samples were applied across the 165 protein spots (98 genes). The mRNA/protein correlation coefficient also varied among proteins with multiple isoforms, indicating potentially separate isoform-specific mechanisms for the regulation of protein abundance. Among the 21 genes with a significant correlation between mRNA and protein, five genes differed significantly between stage I and stage III lung adenocarcinomas. Using a quantitative analysis of mRNA and protein expression within the same lung adenocarcinomas, it was demonstrated that only a subset of the proteins exhibited a significant correlation with mRNA abundance.

Relatively little is known about the regulatory mechanisms controlling the complex patterns of protein abundance and post-translational modification in tumors. Most reports concerning the regulation of protein translation have focused on one or several protein products (Tew et al., Mol. Pharmacol. 50:149 [1996]). Experiments conducted during the course of development of the present invention compared the mRNA and protein expression levels within the same tumor samples. It was demonstrated that 17% (28/165) of the protein spots (21/98 genes) show a statistically significant correlation between mRNA and protein. These proteins appear to represent a diverse group of gene products and include those involved in signal transduction, carbohydrate metabolism, protein modification, cell structure, heat shock and apoptosis. These results suggest that expression of this subset of 165 proteins is likely to be regulated at the transcriptional level in these tissues. The majority of the protein isoforms however, did not correlate with mRNA levels and thus their expression is regulated by other mechanisms. A subset of proteins that demonstrated a negative correlation with the mRNA expression values was also observed; for example alpha-haptoglobin demonstrated a strong negative correlation with its mRNA expression values. This may reflect negative feedback, on the mRNA, the protein, or the presence of other regulatory influences that are not currently understood.

Post-translational modification or processing will result in individual protein products of the same gene migrating to different locations on 2D-PAGE gels (Anderson et al., Electrophoresis 19:1853 [1998]). Experiments conducted during the course of development of the present invention examined 165 protein spots identified in lung adenocarcinomas. Ninety-six protein spots, representing the products of 29 genes, contained at least two protein isoforms. Nineteen of 96 protein spots, representing 12 genes, were shown to have a statistically significant correlation between their protein and mRNA expression, suggesting that the levels of these proteins reflects the transcription of the corresponding genes. Differences in protein-mRNA correlations were found among the individual isoforms of a given protein. For example, of the four OP18 isoforms, three showed a statistically significant correlation between the protein and mRNA expression levels. The lack of relationship for the one isoform, however, indicates that individual protein isoforms of the same gene product can be differentially regulated.

In addition to the analyses of the correlation of mRNA/protein within the same tumor samples, the global relationship between mRNA and the corresponding protein abundance across all 165 protein spots in the lung samples was also tested. A protein and mRNA average value for each gene was generated using all 85 lung tissues samples. A very wide range of normalized average protein and mRNA values was observed. The correlation coefficient generated using this average value data set was −0.025, and even for the 28 protein spots which showed a statistically significant correlation between individual mRNA and proteins, the correlation value was only −0.035. This suggests that it is not possible to predict overall protein expression levels based on average mRNA abundance in lung cancer samples.

A good correlation was reported when the 11 most abundant proteins were examined in yeast (Gygi et al., Mol. Cell Biol. 19:1720 [1999]), suggesting that the level of protein abundance may be a factor that may influence the correlation between mRNA and protein. In the present study, a fairly wide range of mean protein values among 165 protein spots in lung adenocarcinomas was observed and the correlation coefficients also varied from −0.467 to 0.442. A comparison between each protein's mean value and the correlation coefficient generated using all 85 tissue samples did not reveal a strong relationship between the overall protein abundance and the correlation coefficients (r=0.039, p>0.05). Detailed analysis of different subsets of protein abundance also failed to show a correlation between mRNA and protein expression. Thus, in contrast to yeast, a relationship between mRNA-protein correlation coefficient and protein abundance in human lung adenocarcinomas was not observed.

The present invention is not limited to a particular mechanism. Indeed, an understanding of the mechanism is not necessary to practice the present invention. Nonetheless, it is contemplated that the results indicate that the level of protein abundance in lung adenocarcinomas is associated with the corresponding levels of mRNA in 17% (28 proteins) of the total 165 protein spots examined. This was substantially higher than the amount predicted to result by chance alone (which was 5.1) and suggests that a transcriptional mechanism likely underlies the abundance of these proteins in lung adenocarcinomas. Experiments conducted during the course of development of the present invention also demonstrated that the expression of individual isoforms of the same protein may or may not correlate with the mRNA, indicating that separate and likely post-translational mechanisms account for the regulation of isoform abundance. These mechanisms may also account for the differences in the correlation coefficients observed between stage I and stage III tumors, indicating that specific protein isoforms show regulatory changes during tumor progression. The potential to identify specific protein isoforms associated with biological behavior in lung adenocarcinomas adds to the understanding of the regulation of gene products by transcriptional, translational and post-translational mechanisms.

The present invention also demonstrates that the expression of CK7, 8, 18 and 19 in lung adenocarcinoma includes multiple isoforms for each type and suggests regulation may occur at a number of different levels. Several mechanisms for the regulation of cytokeratins have been proposed. These intermediate filaments appear to be assembled and disassembled through rapid phosphorylation and dephosphorylation reactions on specific serine and threonine residues (Nakamura et al., Neurosci Lett 205:91 [1996]; Giasson et al., J Neurochem 70: 1869 [1998]; Escribano and Rozengurt, J Cell Physiol 137: 223 [1988]) can be accelerated by phosphatase inhibition, and can result in the disappearance of the intermediate filament cytoskeleton (Eriksson et al., Proc Natl Acad Sci USA 89:11093 [1992]), Caspase-mediated cleavage leading to cytoskeleton disassembly, formation of pleomorphic cytoplasmic inclusions, and stable cytokeratin fragments (Ku et al., J. Cell Biol. 127:161 [1994]; MacFarlane et al., J Cell Biol 148:1239 [2000]). This early apoptosis-related process has been shown to expose a neo-epitope of CK18 that are not detectable in non-apoptotic epithelial cells (Leers et al., J Pathol. 187:567 [1999]). Massive fragmentation of cytokeratins induced by calcium-dependent proteases was also reported. This rearrangement of cytokeratins has been hypothesized to play a direct role in carcinogenesis by triggering the reorganization of chromatin (Spencer et al., J Biol Chem 273:29093 [1998]), and providing a growth advantage to the transformed cell. Increased vascularization, a characteristic of growing tumors, and the altered permeability of these new blood vessels, was suggested as responsible for the increase in cytokeratin fragments in the serum. It has been also postulated that endogenously produced cytokines may lead to reconstruction of the cytoskeleton in epithelial cells thus increasing the cytokeratins (Bergqvist et al., supra). Increased CK8 expression was reported in breast carcinoma cell lines, where it functions as a plasminogen receptor, activating plasmin, which is important for tumor invasion and cellular migration (Hembrough et al., Biochem J 317:763 [1996]). CK8 was reported to contain antigenic epitopes for antigen CA19-9 (Fujita et al., Br J Cancer 81:769 [1999]), which serves as a ligand for endothelial cell leukocyte molecule-1 (ELAM-1). ELAM-1 mediates cell-cell interactions between platelets and endothelial cells with neutrophils, monocytes, and cancer cells (Takada et al., Biochem Biophys Res Commun 179:713 [1991]; Takada et al., Cancer Res 53:354 [1993]). The present invention is not limited to a particular mechanism. Indeed, an understanding of the mechanism is not necessary to practice the present invention. Nonetheless, it is contemplated that these studies suggest that increased expression of the cytokeratins may reflect changes in the organization of cellular architecture during tumor development and progression. It is contemplated that the variable expression of the specific CK isoforms examined also reflect the regulatory and apoptotic events occurring in lung adenocarcinomas.

A relationship was observed between the mRNA overexpression of the four CK examined in this study (CK7, 8, 18 and 19) and patient outcome in the lung adenocarcinomas. Significant overexpression of many specific isoforms for these proteins was also observed. For example, all five isoforms of CK7 were associated with patient survival. CK7 has an apparent molecular weight range of 49 to 54.3 kDa and a pI range of 5.2 to 5.5, yet all five forms identified by MS were smaller, and not detected by 2D Western blot analysis using two different anti-CK7 antibodies. These results suggest that these smaller CK7 fragments may be missing the epitopes recognized by the CK7 antibodies and may represent proteolytically-modified forms.

Eight CK8 of nearly 15 isoforms were quantified and four found to be significantly over-expressed in lung adenocarcinomas. One isoform (#439) was associated with an unfavorable prognosis and was correlated with CK8 mRNA levels suggesting the abundance of this isoform may be regulated at the level of transcription. Comparison of the immunoreactive proteins between CK7 and CK8 revealed a very similar pattern of fragments with three fragments appearing to overlap. The computer-based matching analysis methods of the present invention revealed many of the isoforms to be unique in their MW and pI values suggesting modifications may differ. Further, the antibodies utilized for Westerns reportedly do not cross-react.

All five CK18 isoforms are over-expressed in lung adenocarcinoma but the native form (529) had the highest frequency of expression. Given the similar MW but differing pI of the CK18 isoforms modification by phosphorylation may underlie these different charged isoforms. The isoforms (#523 and #2324) were significantly correlated with CK18 mRNA also suggesting their abundance may relate to transcriptional regulation. CK19 demonstrated three isoforms of similar MW yet different pI's indicative of potential modification by phosphorylation. CK19 has been documented as a useful tumor marker in lung cancer (Pujol et al., Cancer Res 53:61 [1993]; Pujol et al., Am J Respir Crit Care Med 154:725 [1996]). The most abundant CK19 isoform in both normal and neoplastic lung was #608 which was significantly decreased in lung adenocarcinomas. The two acidic (#609, #1955), and potentially phosphorylated isoforms, were significantly over-expressed in the adenocarcinomas. The most phosphorylated isoform (#1955) was highly elevated (16-fold) in the lung tumors and was associated with unfavorable prognosis.

Correlation analysis of CK mRNAs expression revealed a significant association to the expression of all four CK to each other. This suggests that the mechanisms underlying the increased expression of these proteins in lung tumors may be similar, possibly reflecting the events associated with adenocarcinoma development and progression. Correlation analysis also revealed two other genes that also were significantly correlated in their expression to all four cytokeratin genes. This included liver-specific bHLH-Zip transcription factor and smooth, and non-muscle, myosin light polypeptide 6 genes.

Definitions

To facilitate an understanding of the present invention, a number of terms and phrases are defined below:

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis (e.g., an imaging scan showing a mass) but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “characterizing lung tissue in a subject” refers to the identification of one or more properties of a lung tissue sample (e.g., including but not limited to, the presence of cancerous tissue, the presence of pre-cancerous tissue that is likely to become cancerous, and the presence of cancerous tissue that is likely to metastasize). In some embodiments, tissues are characterized by the identification of the expression of one or more cancer marker genes, including but not limited to, the cancer markers disclosed herein.

As used herein, the term “cancer marker genes” refers to a gene whose expression level, alone or in combination with other genes, is correlated with cancer or prognosis of cancer. The correlation may relate to either an increased or decreased expression of the gene. For example, the expression of the gene may be indicative of cancer, or lack of expression of the gene may be correlated with poor prognosis in a cancer patient. Cancer marker expression may be characterized using any suitable method, including but not limited to, those described in illustrative Examples 1-4 below.

As used herein, the term “decreased survival of said subject” as in “wherein the presence of said marker is indicative of decreased survival of said subject” refers to the correlation of expression or expression level of a cancer marker with the “survival chance” or survival rate of a subject. “Survival chance” or “survival rate” is generally expressed in terms of the likelihood of the subject being alive at a defined time (e.g., five years or ten years) from the time of diagnosis.

As used herein, the term “a risk of developing stage III lung cancer” refers to a statistical likelihood of early (e.g., state I) lung cancer in a subject progressing to stage III lung cancer.

As used herein, the term “a reagent that specifically detects expression levels” refers to reagents used to detect the expression of one or more genes (e.g., including but not limited to, the cancer markers of the present invention). Examples of suitable reagents include but are not limited to, nucleic acid probes capable of specifically hybridizing to the gene of interest, PCR primers capable of specifically amplifying the gene of interest, and antibodies capable of specifically binding to proteins expressed by the gene of interest. Other non-limiting examples can be found in the description and examples below.

As used herein, the term “detecting a decreased or increased expression relative to non-cancerous lung control” refers to measuring the level of expression of a gene (e.g., the level of mRNA or protein) relative to the level in a non-cancerous lung control sample. Gene expression can be measured using any suitable method, including but not limited to, those described herein.

As used herein, the term “detecting a change in gene expression (e.g.,) in said lung cell sample in the presence of said test compound relative to the absence of said test compound” refers to measuring an altered level of expression (e.g., increased or decreased) in the presence of a test compound relative to the absence of the test compound. Gene expression can be measured using any suitable method, including but not limited to, those described in Example 1 below.

As used herein, the term “instructions for using said kit for detecting cancer in said subject” includes instructions for using the reagents contained in the kit for the detection and characterization of cancer in a sample from a subject. In some embodiments, the instructions further comprise the statement of intended use required by the U.S. Food and Drug Administration (FDA) in labeling in vitro diagnostic products. The FDA classifies in vitro diagnostics as medical devices and requires that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use. Where applicable, photographs or engineering drawings should be supplied; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; 8) Any additional information regarding the in vitro diagnostic product requested that is necessary for the FDA to make a substantial equivalency determination. Additional information is available at the Internet web page of the U.S. FDA.

As used herein, the term “instructions for using said kit for predicting survival in said subject” includes instructions for using the reagents contained in the kit for the predicting a “survival rate” or “survival chance” in a subject. In some embodiments, the instructions further comprise the statement of intended use required by the U.S. Food and Drug Administration (FDA) in labeling in vitro diagnostic products (See e.g., above description).

As used herein, the term “lung cancer expression profile map” refers to a presentation of expression levels of genes in a particular type of lung tissue (e.g., primary, metastatic, and pre-cancerous lung tissues). The map may be presented as a graphical representation (e.g., on paper or on a computer screen), a physical representation (e.g., a gel or array) or a digital representation stored in computer memory. Each map corresponds to a particular type of lung tissue (e.g., primary, metastatic, and pre-cancerous) and thus provides a template for comparison to a patient sample.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “providing a prognosis” refers to providing information regarding the impact of the presence of cancer (e.g., as determined by the diagnostic methods of the present invention) on a subjects future health (e.g., expected morbidity or mortality, the likelihood of getting cancer, and the risk of metastasis).

As used herein, the term “post surgical tumor tissue” refers to cancerous tissue (e.g., lung tissue) that has been removed from a subject (e.g., during surgery).

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention.

As used herein, the term “initial diagnosis” refers to results of initial cancer diagnosis (e.g. the presence or absence of cancerous cells). An initial diagnosis does not include information about the stage of the cancer of the risk of metastasis.

As used herein, the term “biopsy tissue” refers to a sample of tissue (e.g., lung tissue) that is removed from a subject for the purpose of determining if the sample contains cancerous tissue. In some embodiment, biopsy tissue is obtained because a subject is suspected of having cancer. The biopsy tissue is then examined (e.g., by microscopy) for the presence or absence of cancer.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Upregulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (Kacian et al, Proc. Natl. Acad. Sci. USA 69:3038 [1972]). Other nucleic acids will not be replicated by this amplification enzyme. Similarly, in the case of T7 RNA polymerase, this amplification enzyme has a stringent specificity for its own promoters (Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target.” In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein, the term “target,” refers to the region of nucleic acid bounded by the primers. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (e.g., the oligonucleotide or polynucleotide may be single-stranded), but may contain both the sense and anti-sense strands (e.g., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 [1973]), has been modified by several groups to optimize conditions for particular types of cells. The art is well aware of these numerous modifications.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp.16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

Detailed Description of the Invention

The present invention relates to compositions and methods for cancer diagnostics, including but not limited to, cancer markers. In particular, the present invention provides gene expression profiles associated with lung cancers. Accordingly, the present invention provides method of characterizing lung tissues, kits for the detection of markers, as well as drug screening and therapeutic applications.

I. Markers for Lung Cancer

The present invention provides markers whose expression is specifically altered in cancerous lung tissues. Such markers find use in the diagnosis and characterization of lung cancer.

A. Identification of Markers

The methods of the present invention (See e.g., Examples 1-4) were used to identify clusters of genes that were up or down regulated in lung cancer. Several markers were identified that correlated with the presence, stage or prognosis of lung cancer (e.g., including, but not limited to, GRP-58, PSMC, VIM, SOD, TPI, AOE372, ATP5D, B4GALT, Ppase, GRP58, GSTM4, P4HB, TPI, UCHL1, CK19, CK7, and CK8). The markers of the present invention find use in a variety of applications, including, but not limited to, diagnostic, research, and clinical applications.

B. Detection of Markers

In some embodiments, the present invention provides methods for detection of expression of cancer markers (e.g., lung cancer markers). In preferred embodiments, expression is measured directly (e.g., at the RNA or protein level). In some embodiments, expression is detected in tissue samples (e.g., biopsy tissue). In other embodiments, expression is detected in bodily fluids (e.g., including but not limited to, plasma, serum, whole blood, and urine). The present invention further provides panels and kits for the detection of markers. In preferred embodiments, the presence of a cancer marker is used to provide a prognosis to a subject. For example, the detection of certain markers (e.g., CK19, CK7, and CK8) is indicative of decreased rates of survival. The information provided is also used to direct the course of treatment. For example, if a subject is found to have a marker indicative of a highly metastasizing tumor, additional therapies (e.g., experimental therapies) can be started at a earlier point when they are more likely to be effective (e.g., before metastasis).

The present invention is not limited to the markers described above. Any suitable marker that correlates with cancer or the progression of cancer may be utilized, including but not limited to, those described in the illustrative examples below (e.g., Examples 1-4). Any suitable method may be utilized to identify and characterize cancer markers suitable for use in the methods of the present invention, including but not limited to, those described in illustrative Examples 1-4 below.

In some embodiments, the present invention provides a panel for the analysis of a plurality of markers. The panel allows for the simultaneous analysis of multiple markers correlating with survival, carcinogenesis and/or metastasis. For example, a panel may include markers identified as correlating with cancerous tissue, metastatic cancer, localized cancer that is likely to metastasize, pre-cancerous tissue that is likely to become cancerous, and pre-cancerous tissue that is not likely to become cancerous. In other embodiments, panels include markers correlating with survival rates. Depending on the subject, panels may be analyzed alone or in combination in order to provide the best possible diagnosis and prognosis. Markers for inclusion on a panel are selected by screening for their predictive value using any suitable method, including but not limited to, those described in the illustrative examples below.

In other embodiments, the present invention provides an expression profile map comprising expression profiles of cancers of various stages or prognoses (e.g., likelihood of future metastasis). Such maps can be used for comparison with patient samples. Maps maybe compared using any suitable method (e.g., including but not limited to, by computer comparison of digitized data). The comparison data is used to provide diagnoses and/or prognoses to patients.

1. Detection of RNA

In some preferred embodiments, detection of lung cancer markers (e.g., including but not limited to, those disclosed herein) is detected by measuring the expression of corresponding mRNA in a tissue sample (e.g., lung tissue). In other embodiments, expression of mRNA is measured in bodily fluids, including, but not limited to, blood, serum, mucus, and urine. In some preferred embodiments, the level of mRNA expression in measured quantitatively. RNA expression may be measured by any suitable method, including but not limited to, those disclosed below.

In some embodiments, RNA is detected by Northern blot analysis. Northern blot analysis involves the separation of RNA and hybridization of a complementary labeled probe. In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes.

In still further embodiments, RNA (or corresponding cDNA) is detected by hybridization to a oligonucleotide probe). A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

2. Detection of Protein

In other embodiments, gene expression of cancer markers is detected by measuring the expression of the corresponding protein or polypeptide. In some embodiments, protein expression is detected in lung tissue. In other embodiments, protein expression is detected in bodily fluids. In some embodiments, the level of protein expression is quantitated. Protein expression may be detected by any suitable method. In some embodiments, proteins are detected by the immunohistochemistry methods disclosed herein (e.g., in Examples 1-4). In other embodiments, proteins are detected by their binding to an antibody raised against the protein. The generation of antibodies is described below.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference

3. Kits

In yet other embodiments, the present invention provides kits for the detection and characterization of lung cancer. In some embodiments, the kits contain antibodies specific for a cancer marker, in addition to detection reagents and buffers. In other embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results.

II. Antibodies

The present invention provides isolated antibodies. In preferred embodiments, the present invention provides monoclonal antibodies that specifically bind to an isolated polypeptide comprised of at least five amino acid residues of the cancer markers described herein (See e.g., the markers described in illustrative Examples 1-4 below). These antibodies find use in the diagnostic and therapeutic methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a cancer marker of the present invention (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

III. Drug Screening

In some embodiments, the present invention provides drug screening assays (e.g., to screen for anticancer drugs). The screening methods of the present invention utilize cancer markers identified using the methods of the present invention (e.g., including but not limited to, GRP-58, PSMC, VIM, SOD, TPI, AOE372, ATP5D, B4GALT, Ppase, GRP58, GSTM4, P4HB, TPI, UCHL1, CK19, CK7, and CK8). For example, in some embodiments, the present invention provides methods of screening for compound that alter (e.g., increase or decrease) the expression of cancer marker genes. In some embodiments, candidate compounds are antisense agents (e.g., oligonucleotides) directed against cancer markers. See Section IV below for a discussion of antisense therapy. In other embodiments, candidate compounds are antibodies that specifically bind to a cancer marker of the present invention.

In one screening method, candidate compounds are evaluated for their ability to alter cancer marker expression by contacting a compound with a cell expressing a cancer marker and then assaying for the effect of the candidate compounds on expression. In some embodiments, the effect of candidate compounds on expression of a cancer marker gene is assayed for by detecting the level of cancer marker mRNA expressed by the cell. mRNA expression can be detected by any suitable method. In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein.

IV. Cancer Therapies

In some embodiments, the present invention provides therapies for cancer (e.g., lung cancer). In some embodiments, therapies target cancer markers (e.g., including but not limited to, GRP-58, PSMC, VIM, SOD, TPI, AOE372, ATP5D, B4GALT, Ppase, GRP58, GSTM4, P4HB, TPI, UCHL1, CK19, CK7, and CK8).

A. Antisense Therapies

In some embodiments, the present invention targets the expression of cancer markers. For example, in some embodiments, the present invention employs compositions comprising oligomeric antisense compounds, particularly oligonucleotides (e.g., those identified in the drug screening methods described above), for use in modulating the function of nucleic acid molecules encoding cancer markers of the present invention, ultimately modulating the amount of cancer marker expressed. This is accomplished by providing antisense compounds that specifically hybridize with one or more nucleic acids encoding cancer markers of the present invention. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of cancer markers of the present invention. In the context of the present invention, “modulation” means either an increase (stimulation) or a decrease (inhibition) in the expression of a gene. For example, expression may be inhibited to potentially prevent tumor proliferation.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a cancer marker of the present invention. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂— [wherein the native phosphodiester backbone is represented as—O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]2, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrirnidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2. degree ° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-S-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

B. Genetic Therapies

The present invention contemplates the use of any genetic manipulation for use in modulating the expression of cancer markers of the present invention. Examples of genetic manipulation include, but are not limited to, gene knockout (e.g., removing the cancer marker gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. appl. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

C. Antibody Therapy

In some embodiments, the present invention provides antibodies that target lung tumors that express a cancer marker of the present invention (e.g., GRP-58, PSMC, VIM, SOD, TPI, AOE372, ATP5D, B4GALT, Ppase, GRP58, GSTM4, P4HB, TPI, UCHL1, CK19, CK7, and CK8). Any suitable antibody (e.g., monoclonal, polyclonal, or synthetic) may be utilized in the therapeutic methods disclosed herein. In preferred embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against a cancer marker of the present invention (e.g., GRP-58, PSMC, VIM, SOD, TPI, AOE372, ATP5D, B4GALT, Ppase, GRP58, GSTM4, P4HB, TPI, UCHL1, CK19, CK7, and CK8), wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeted a cancer marker of the present invention (e.g., GRP-58, PSMC, VIM, SOD, TPI, AOE372, ATP5D, B4GALT, Ppase, GRP58, GSTM4, P4HB, TPI, UCHL1, CK19, CK7, and CK8). Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

D. Pharmaceutical Compositions

The present invention further provides pharmaceutical compositions (e.g., comprising the therapeutic compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more antisense or antibody compounds and (b) one or more other chemotherapeutic agents that function by a different mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

V. Transgenic Animals Expressing Cancer Marker Genes

The present invention contemplates the generation of transgenic animals comprising an exogenous cancer marker gene of the present invention or mutants and variants thereof (e.g., truncations or single nucleotide polymorphisms). In preferred embodiments, the transgenic animal displays an altered phenotype (e.g., increased or decreased presence of markers) as compared to wild-type animals. Methods for analyzing the presence or absence of such phenotypes include but are not limited to, those disclosed herein. In some preferred embodiments, the transgenic animals further display an increased or decreased growth of tumors or evidence of cancer.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the microinjection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); 1 or L (liters); ml (milliliters); μl (microliters); cm (centimeters); mm (millimeters); μm (micrometers); nm (nanometers); and ° C (degrees Centigrade).

### Example 1

Gene Expression Profiling of Lung Cancer

The Example describes the expression profiling of lung cancer to identify genes that exhibit up-regulated expression in lung cancer.

A. Methods

Patient Population

Sequential patients seen by the General Thoracic Surgery Section at the University of Michigan Hospital between May 1994 and July 2000 for resection of stage I or stage III lung adenocarcinoma were evaluated for inclusion in this study. Consent was received from all patients and the project approved by the Institutional Review Board. Patients' medical records were reviewed and patient identifiers coded to protect confidentiality. Tumor and adjacent non-neoplastic lung tissue were obtained immediately at the time of surgery and carried to the laboratory in Dulbecco's modified Eagle medium (GibcoBRL, Gathersburg, Md.) on ice. Tumor samples were obtained from the periphery of resected lung carcinomas, embedded in OCT (Miles Scientific, Naperville, Ill.) for cryostat sectioning, frozen in liquid nitrogen and stored at −80° C. Hematoxylin-stained cryostat sections (5 μm) of tissue to be utilized for mRNA isolation were evaluated by a study pathologist and compared with routine H&E sections of the same tumors. All specimens were primary adenocarcinomas from which the regions chosen for analysis contained a tumor cellularity greater than 70%. None of the tumors were of mixed histology (e.g., adenosquamous), potential metastatic origin as indicated by previous tumor history, extensive lymphocytic infiltration, or fibrosis. Tumors were histopathologically divided into two broad categories: bronchial-derived, if they exhibited invasive features with architectural destruction, and bronchioloalveolar, if they exhibited preservation of the lung architecture. Tumors were further sub-classified as mucinous, papillary, or clear cell depending on the predominant histologic appearance. All stage I patients received only surgical resection with complete intra-thoracic nodal dissection and no other treatments. Stage III patients received surgical resection plus chemotherapy and radiotherapy.

Gene Expression Profiling

RNA isolation, cDNA synthesis and gene expression profiling were performed as described (Giordano et al., Am. J. Pathol., 159:1231 [2001]). A Unigene cluster was assigned to each of the Affymetrix probe sets to obtain gene specific annotation (gene name and Unigene description). Probe set sequences provided by Affymetrix were aligned to each of the sequences in the Unigene Repository at the National Center for Biotechnology Information (available at the Internet web site of the National Institutes of Health) using the Basic Local Alignment Search Tool (BLAST) algorithm (Altscul et al., J. Mol. Biol., 215:403 [1990]). The priority for cluster assignment was as follows: 1) perfect match to sequence, or 2) an E value less than 10⁻⁵⁰. Cluster identity was assigned the value “NULL” if none of these criteria were achieved. Replicate experiments of selected individual samples were performed as a quality control.

Northern Blot Analysis

Ten micrograms of total cellular RNA was separated in 1.2% agarose-formaldehyde gels and vacuum-transferred to Gene Screen Plus (NEN Life Science Products, Boston, Mass.). Individual cDNA image clones for human insulin-like growth factor binding protein 3 (IGFBP3; clone 1407750), lactate dehydrogenase A (LDHA; clone 2420241), and cystatin C (CTS3; clone 949938) were obtained from Research Genetics (Huntsville, Ala.). Each clone was verified by DNA sequencing and the inserts removed with appropriate restriction enzymes and then utilized for ³²P-labeling using a random primer kit (GibcoBRL). The human H4 histone cDNA and the 28S ribosomal RNA 26-mer oligonucleotide probes were prepared and labeled as previously described (Hanson et al., Exp. Lung Res., 17:371 [1991]). Hybridization and washing conditions are as described (Hanson et al., supra), and the resulting signals were obtained and quantified using phosphor-image analysis (Molecular Dynamics, Sunnyvale, Calif.).

Mutational Analysis of K-ras

Genomic DNA was isolated from each tumor sample using the organic phase of the Trizol reagent from which the total RNA was obtained. The procedure utilized is as described by the manufacturer (GibcoBRL). Fifty ng of each tumor DNA sample was subjected to PCR amplification using the primers that encompass codons 12 and 13 of the K-ras gene. The sequences of forward and reverse primers are 5′ TATAAGGCCTGCTGAAAAT 3′ (SEQ ID NO:1) and 5′CCTGCACCAGTAATATGC 3′ (SEQ ID NO:2), respectively. A portion of the 165 bp PCR product was verified for each tumor sample by agarose gel electrophoresis (GibcoBRL) and visualized by ethidium bromide staining. The remainder of each PCR reaction was purified using Microcon-PCR Filter Unit (Millipore Corporation, Bedford, Mass.) according to the manufacturer's protocol. Two nanograms of purified PCR products containing the exon 1 of the K-ras gene were then subjected to thermal cycle sequencing with an internal nested primer (5′ AGGCCTGCTGAAAATGACT 3′ (SEQ ID NO:3)) using Thermo Sequenase Radiolabeled Terminator Cycle Sequencing Kit (USB Corporation, Cleveland, Ohio) following the protocol from the manufacturer. Sequencing products were resolved in 8% urea PAGE gels, dried, exposed to Phosphor-Image screens and visualized using a Phosphor-Image scanner (Molecular Dynamics, Sunnyvale, Calif.). Mutations were determined by comparing each tumor DNA sequence of K-ras 12th and 13th codon to its wild-type sequence (GGTGGC).

Gene Amplification Analysis

Eleven genes were selected for the analysis of genomic alterations. Primers were designed using PRIMERSELECT 4.05 Windows 32 software (DNASTAR, Inc., Madison, Wis.), avoiding pseudogenes or potential homologous regions. Forward and reverse primers for the genes are listed in Table 2. Quantitative genomic-PCR was then applied as previously described (Lin et al., Cancer Res. 60:1341 [2000]). Each selected gene was co-amplified with GAPDH as an internal control. Equal amounts of genomic DNA from tumor and normal tissue from the same patient were used. The forward primers of GAPDH and the test fragments were end-labeled with [³²P-γ]-ATP (NEN Life Science Products) using T4 polynucleotide kinase (New England BioLabs, Beverly Mass.). PCR was conducted with a 40 ng template in 25 μl of total reaction volume using Taq polymerase (Promega, Madison, Wis.) and subjected to 20 reaction cycles. The PCR products were then resolved on 8% denaturing polyacrylamide gels. The signal ratios (Ts/c: Ns/c) for both the tumor (Ts/c, tumor tested gene fragment/tumor GAPDH fragment) and normal DNA samples (Ns/c, normal tested gene fragment/normal GAPDH fragment) were determined using ImageQuant software (Molecular Dynamics).

Immunohistochemical Staining

The H&E stained slides of all primary lung tumors used in this study were examined by a study pathologist and the diagnosis of adenocarcinoma confirmed. Areas of the tumor that best represented the overall morphology were selected, and a tissue microarray (TMA) block was constructed according to the method of Kononen (Konenen et al., Nature Med. 4:844 [1998]) to facilitate the examination of a large number of cases. Immunohistochemistry (IHC) was performed using both routine sections and sections from the TMA block. Deparaffinized sections, along with sections from a commercially available normal tissue TMA block (NO50, Clinomics Laboratories, Pittsfield, Mass.) were microwave pretreated in citric acid buffer to retrieve antigenicity. The sections were incubated with blocking solution for 60 min at room temperature prior to being exposed to the following concentrations of primary antibodies: anti-p53 (M-7001, 1.0 μg/ml), KRT19 (M0772, 1.3 μg/ml), erbB2 (A0485, 2.5 μg/ml), CK7 (OV-TL 12/30, 880 μg/ml, protease pretreatment, Dako Corporation, Carpinteria, Calif.); IGFBP-3, (SC-9028, 1.0 μg/ml), HSP-70 (SC-1060, 2.0 μg/ml), VEGF (A-20, 2.0 μg/ml), Cdc6 (180.2, 2.0 μg/ml), BMP2 (A-20, 2.0 μg/ml, Santa Cruz Biotechnology, Santa Cruz, Calif.), cystatin C (Upstate Biotechnology, Lake Placid, N.Y., 06-458, 5 μg/ml; SlOOP (2.5 μg/ml), FADD (2.5 μg/ml), CASP4 (66171A, 0.9 μg/ml), Crk (2.5 pg/ml), BD Biosciences PharMingen, San Diego, Calif., which recognizes both crkI and crkII proteins, products of the crk gene arising from alternative splicing; CRT18 (NCL-CK18), Novocastra Laboratories Ltd, UK. Antibodies were incubated with the tissue sections overnight at 4° C. Primary antibody-antigen complexes were visualized by the immunoglobulin enzyme bridge technique using Vector ABC-kit (Vector Laboratories, Burlingame, Calif.). The enzyme substrate was either 3,3′ diaminobenzidine tetrachloride (most antibodies), or BCIP/NBT (IGFBP3). The sections were weakly counter-stained with either hematoxylin or nuclear fast red. Immunohistochemically stained slides were reviewed and evaluated independently by two pathologists. The nuclear accumulation of the p53 protein was defined when present in the nuclei of >50% of the tumor.

Statistical Methods

A detailed examination and distributional testing of the raw perfect match (PM) and mismatch (MM) data using a random sample of genes on the HUGeneFL chip for a random sample of the tumors indicated that a trimmed mean estimate of a gene's expression level was a more robust measure of gene expression than the Affymetrix-calculated average difference. For this analysis, trimmed means were computed by dropping the top and bottom 25% of the PM-MM data for the set of oligonucleotides corresponding to individual genes, before averaging the PM-MM's. Array to array variation in the overall distribution of gene expression values detected by quantile-quantile plots was removed by applying a quantile normalization using a linear spline as a monotone transformation. The gene expression profile of each tumor was normalized to the median gene expression profile for the entire sample. More details of these procedures can be found in Giordano et al (supra). Features on the oligonucleotide arrays representing the genes in the individual tumors found as outliers were carefully reviewed to confirm expression levels and exclude artifacts. T-tests were used to identify differences in mean gene expression levels between comparison groups. Agglomerative hierarchical clustering (Johnson, R. & Wichem D. W. In: Applied Multivariate Statistical Analysis. Prentice Hall: New Jersey, pp. 543-578 (1988) was applied using the average linkage method to investigate whether there was evidence for natural groupings of tumor samples based on correlations between gene expression profiles. To investigate the robustness of the clustering inference, gene expression values were perturbed by adding random Gaussian error of magnitude obtained from a duplicate sample to each data point and then reclustered to determine concordance in the tumor's class membership. Pearson chi-square and Fisher's Exact tests were used to assess whether cluster membership was associated with physical and genetic characteristics of the tumors.

To determine whether gene expression profiles were associated with variability in survival times, two separate but complementary approaches were used. In the first approach the 86 tumors were randomly assigned to equivalent training and testing sets consisting of equal numbers of stage I and III tumors in order to validate a novel risk index function that captured the effect of many genes at once. The top 10, 20, 50 and 75 genes most significant for survival in the training set were used to create a risk index. The risk index was defined as a linear combination of the gene expression values for the top genes identified by univariate Cox proportional hazard regression modeling (Cox, J. R. Stat. Soc. 34:187 [1972]) weighted by their estimated regression coefficients. The distribution of risk index values calculated in the training set was examined to determine an appropriate cut point to distinguish high and low risk. A continuum of cutpoints (ranging from the 45^{th} to 90^{th} percentile) was examined. The results are reported for the 60^{th} percentile cut point as the 50^{th} and 70^{th} percentiles gave approximately the same results. Using the risk index function and the high low risk cutpoint estimated in the training set, the risk index value for each test case was calculated and used to assign each set to a high or low risk group. Kaplan-Meier (Kaplna and Meier, J. Am. Stat. Assoc. 53:457 [1958]) survival plots and log rank tests were then used to assess whether the risk index assignment was validated in the test set. In the second approach, another common training-testing method known as cross-validation (Stone, Biometrika 64:29 [1977]) was used to more robustly identify the genes associated with survival. Briefly, a leave-one-out cross validation procedure in which 85 of the 86 tumors (the training set) was used to identify genes that were univariately associated with survival. The top 50 genes were used to create a risk index in each training sample (as described above) that would be tested later on the single tumor (the test case) that had been removed earlier. Repeating this training-testing procedure 86 independent times, the risk index of the test case was compared with the risk index cutpoint estimated in the training cases. Each test case was designated as high and low risk again using 60^{th} percentile cutpoint estimated in the training set. Survival curves were investigated using Kaplan-Meier survival plots and log rank tests. Leave-five-out and leave-ten-out, training-testing, cross-validation were also performed but the results didn't noticeably differ from the results of the leave-one-out strategy. After conducting the training-testing procedure, genes were ranked based on the number of times they had a significant association at an alpha=0.01 level of significance in any one of the 86 training-testing cross validation. The top 100 of these genes are reported in Table 1.

An additional raw data set (Bhattacharjee et al., PNAS, 98:13790 [2001]) was obtained to validate the results. A total of 84 lung adenocarcinoma (stages I-III) including 62 stage I tumors were obtained. The array was a HG_U95Av2 array from Affymetrix. A stage I adenocarcinoma was selected as a standard, and probe pairs with PM-MM<200 discarded. Probe-sets intensities were computed in the same manner as the Michigan samples. Quantile normalization to the standard was performed using 99 evenly spaced quantiles and numbers from replicated arrays for the same tumor averaged. Probe-sets of interest on the HG_U95Av2 most representing the 50 probe-sets of interest on the HuGeneFL arrays were determined byjoining probe-sets with identical Unigene Cluster identifiers.

B. Results

Hierarchical Clustering of Gene Expression Profiles Yields Three Subsets of Tumors

Gene expression profiles were generated for 86 primary lung adenocarcinomas, including 67 stage I and 19 stage III tumors as well as non-neoplastic lung samples from ten of the patients, using HuGeneFL Affymetrix oligonucleotide arrays. Selected replicate experiments of individual samples indicated very high correlation coefficients and excellent reproducibility. Transcript abundance was determined using a custom algorithm and the data set was trimmed to remove genes that were not expressed or expressed at very low levels, i.e., genes were excluded from further analysis if the measure of their 75 percentile value was <100. Trimming in this manner, although potentially resulting in the loss of some informative genes, decreased the possibility that the agglomerative hierarchical clustering algorithm was strongly influenced by genes with little or no expression in these samples. Hierarchical clustering with the resulting 4966 genes yielded three clusters of tumors (FIG. 1). All 10 non-neoplastic lung samples clustered tightly together within Cluster 1. The relationships between cluster and patient and tumor characteristics was also examined (FIG. 1). A significant relationship was observed between cluster and tumor stage (P=0.030) and between cluster and tumor differentiation (P=0.010). Cluster 1 contained the greatest percentage (42.8%) of well-differentiated tumors, followed by Cluster 2 (27%) and Cluster 3 (4.7%). Cluster 3 contained the highest percentage of both poorly differentiated (47.6%) and stage III tumors (42.8%), yet contained three (14.3%) moderately differentiated and one (5%) well-differentiated stage I tumor. 11 stage I tumors were present in Cluster 3 suggesting a common gene expression profile for this subset of stage I and stage III tumors.

The average age was 68.1 and 64.5 years and the percentage of smokers was 88.9 and 89.5 for patients with stage I and stage III tumors, respectively. Marginally significant associations between cluster and smoking history were observed (P=0.06). A significant relationship between histopathologic classification and cluster was only discernable for bronchioloalveolar adenocarcinomas (BA) which were only present in Clusters 1 and 2 (P=0.0055), and comprising 35.7% and 12.3% of tumors for Clusters 1 and 2, respectively.

Heterogeneity in gene expression profiles based on the trimmed data set among normal lung samples, and stage I and stage III adenocarcinomas was examined by calculating correlation coefficients between all pairs of samples. In contrast to normal lung samples that displayed very similar gene expression profiles (median correlation=0.9), both stage I and III lung tumors demonstrated much greater heterogeneity in their expression profiles with lower correlation coefficients (median values=0.82, and 0.79, respectively).

Analysis of a subset of genes using northern blot and immunohistochemistry of the 4966 genes examined, 967 genes differed significantly between stage I and III adenocarcinomas, a number in excess of that expected by chance alone (4966 genes*0.05 alpha level=248 genes expected by chance). Three of these genes were selected to verify the microarray expression data. The mRNA from 20 of the normal lung and tumor samples was examined by northern blot hybridization with cDNA probes for the IGFBP3, cystatin C and LDH-A genes (FIG. 2a). Two probes not represented on the microarrays were utilized as controls. The H4 histone gene was examined as a potential index of overall cell proliferation and the 28S ribosomal RNA gene was used a control for sample loading and transfer. The relative amounts of IGFBP3, cystatin C and LDH-A mRNA, as measured by integrated phosphor imager-based signals, strongly correlated with microarray-based measures (FIG. 2b). In both assays, IGFBP3 and LDH-A mRNA levels increased from stage I to stage III adenocarcinomas, and were higher than normal lung. Cystatin C mRNA levels were more variable but relatively had greater expression in normal lung than tumors. These results suggest that the oligonucleotide microarrays provide reliable measures of gene expression. The tumors showed slightly greater H4 histone expression than the normal lung, likely reflecting increased proliferation of tumor cells.

Immunohistochemical analysis was performed for IGFBP3, cystatin C and HSP70 to determine whether mRNA overexpression was reflected by an increase of their corresponding proteins in tumors. Immunoreactivity for both IGFBP-3 and HSP-70 was detected in the cytoplasm of the adenocarcinomas, with little detectable reactivity in the stromal or inflammatory cells. Cystatin C immunoreactivity was detected in alveolar pneumocytes and intra-alveolar macrophages in non-neoplastic lung parenchyma and also consistently in the cytoplasm of neoplastic cells.

Gene Expression Profiles Predict Survival

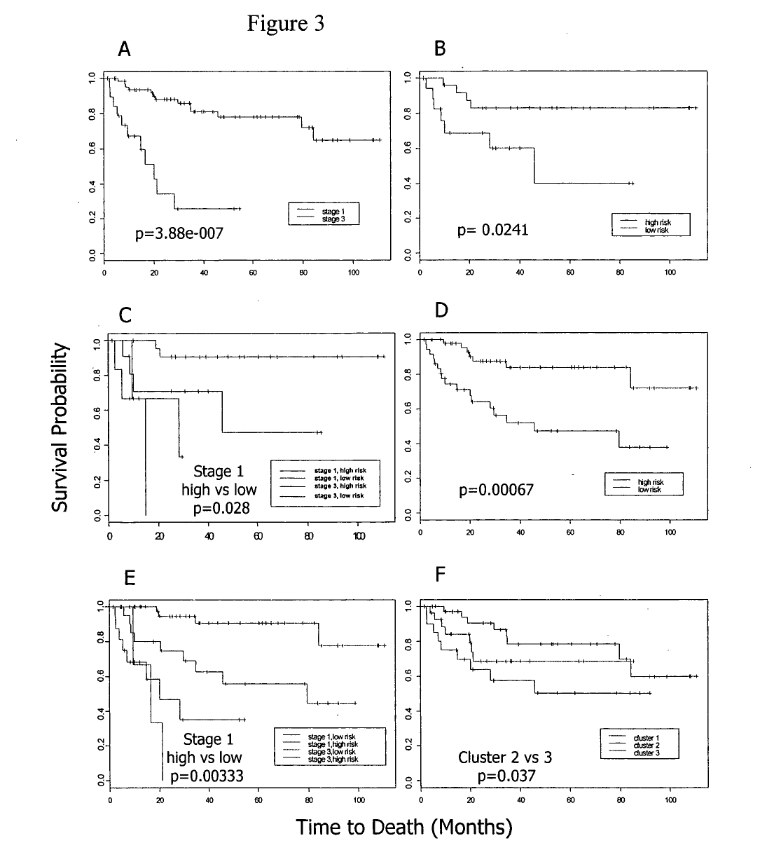

As expected, Kaplan-Meier survival curves (FIG. 3a) and log rank tests indicated a significantly poorer survival among stage III compared to stage I adenocarcinomas (P=3.38e-007). Two statistical approaches were utilized to determine whether gene expression profiles could predict survival using the 4966 gene data set. In one approach equal numbers of randomly assigned stage I and stage III tumors constituted training (n=43) and testing (n=43) sets. In the training set the top 10, 20, 50 or 75 genes were used to create risk indices that were evaluated for their association with survival using the 50^{th}, 60^{th}, or 70^{th} percentile cutpoints to categorize patients into high/low groups. The results were similar across cutpoints but the 50-gene risk index had the best overall association with survival. Conservatively choosing the 60^{th} percentile cutpoint from the training set, this risk index and cutpoint was then applied to the testing set. The top 50 gene risk index correctly identified low and high risk individuals within the independent testing set (P=0.024) (FIG. 3b). 11 stage I tumors were included in the high-risk subgroup. When this risk assignment was then examined conditional for stage (FIG. 3c), a low and high-risk group among stage I tumors were found to differ significantly (P=0.028) in their survival. Stage III low and high-risk groups were not significantly different (P=0.634).

Identification of a Robust Set of Survival Genes

Although predictive of patient survival, a single training-testing set may not provide the most robust set of genes for further examination due to random sampling issues. Therefore we also used a leave-one-out cross validation approach to identify genes associated with survival from all 86 tumor samples. A risk index was first developed in each training set, and then applied the risk index to the test case held out from the full set of tumors and assigned the held out tumor to the high or low risk groups (FIG. 3d). The high and low risk subgroups determined in the test cases differed significantly in their overall survival (P=0.0006). Among the larger group of stage I lung adenocarcinomas, the low risk (n=46) and high-risk (n=21) groups had markedly different survival (P=0.003) (FIG. 3e). Table 1 lists the cumulative top 100 genes derived from this cross-validated sample. Twenty-four of the 50 genes predictive of survival in the 43 tumor test set were also present in the cross-validated top 100 gene list It was also noted that many of the stage I patients in the high-risk subgroup (FIG. 3e) were present in Cluster 3 (FIG. 1). Kaplan-Meier analysis (FIG. 3f) demonstrated a significantly worse survival (P=0.037) for patients in Cluster 3 relative to patients in Cluster 2 and approaching significance for Cluster 1 and 2 combined (P=0.06). This further indicates the important relationship between gene expression profiles and patient survival, independent of stage.

40% of stage I and 57.8% of stage III tumors had 12^{th} or 13^{th} codon K-ras gene mutations. Those patients with tumors containing K-ras mutations showed a trend of poorer survival, but this difference did not reach statistical significance among all patients (P=0.25), between patients within tumor clusters (P=0.41), or when analyzed separately among stage I (P=0.22) and stage III (P=0.53) patients. Nuclear accumulation of p53 was detected in 17.9% stage I and in 22.2% stage III tumors. No significant relationship was observed for p53 staining and patient survival, cluster, or tumor stage. Confirmation of the Risk-Index using an independent set of adenocarcinomas The ability of the 50 gene risk index to predict survival in lung adenocarcinomas was tested using oligonucleotide gene expression data obtained from a completely independent (Massachusetts-based) sample of 84 lung adenocarcinomas (62 stage I, 14 stage II and 8 stage III; ref. 21; dataset A; www.genome.wi.mit.edu/MPR/lung). Criteria for inclusion of the tumors in the analysis were 40% or greater tumor cellularity, no mixed histology (i.e., adenosquamous) and patient survival information. To obtain comparative gene expression measures between the two data sets, gene sequences present on the Affyrnetrix array (U95) and HuGeneFL arrays were examined and expression data for the 50 top cross-validation genes for all 84 Massachusetts samples obtained. When the risk assignment was examined on these 84 samples, employing the identical cut-point used for the Michigan-based 86 lung samples, a low and high-risk group was observed (P=0.003). Most importantly, among the 62 stage I tumors a high and low-risk group were observed that differed very significantly (P=0.006) in their survival.

Survival Genes Display Graded and Outlier Expression Patterns

A statistical and graphical analysis of the 100 survival-related genes identified by cross-validation methods (Table 1), clustered against all 86 tumors revealed individual tumors with substantially elevated expression in both a limited and larger number of genes (FIG. 4a). Among these genes two distinct patterns of expression related to patient survival were observed. One pattern, designated as an “outlier”, included genes showing substantially elevated expression (greater than five times the interquartile range among all samples) present in one or few tumors while the other pattern, designated “graded”, was characterized by continuously distributed expression with survival for most tumors (FIG. 4b). The erbB2 and Reg1A genes are shown as examples of outlier expression patterns and S100P and crk genes as graded patterns. The number of outliers per person identified in the top 100 genes and plotted according to survival times and events is shown in FIG. 4c. Both stage I and stage III lung adenocarcinomas showed outlier gene patterns and ten tumors contained 3 or more outlier genes. An analysis of the Michigan-based risk index using top cross-validated survival genes identified a low and high-risk group in an independent cohort of 84 Massachusetts-based lung adenocarcinomas that are significantly different (P=0.003). Among the 62 stage I lung adenocarcinoma in the Massachusetts sample, the high risk and low groups were observed to differ significantly (p=0.006).

Gene amplification is one mechanism that may result in increased gene expression. Nine genes with outlier expression patterns (erbB2, SLC1A6, Wnt 1, MGB1, Reg1A, AKAP12, PACE, CYP24, KYNU), and one gene with a graded expression pattern (KRT18), were examined using quantitative genomic PCR to detect increased genomic copy number. Gene amplification of erbB2 (17q12) was detected in tumor L94, the tumor with the highest erbB2 mRNA expression (FIG. 5). Gene amplification was not detected for any of the other seven tested genes that also showed highly elevated expression in tumor L94, as well as in other tumors. The two genes most frequently demonstrating the outlier pattern in these lung adenocarcinomas were KYNU and CYP24, being present in 10 and 9 tumors respectively. CYP24 has been described as a gene amplified and overexpressed in breast cancer, and these results indicate elevated expression in lung adenocarcinoma.

To determine whether the graded or outlier gene expression patterns are also observed at the protein expression level, ten of the 100 top survival genes (Table 1) for which specific antibodies were available were chosen for immunohistochemical analysis using lung tumor arrays from this study. Expression of membrane erbB2 protein was substantially increased in the erbB2-amplified tumor L94 and very low, to undetectable levels of expression was present in other tumors, consistent with mRNA expression measures. CDC6 protein expression was also substantially higher in tumor L94, consistent with mRNA levels. Expression of VEGF and S100P, as well as KRT18, KRT7, and FADD protein, was located within the lung tumor cells and consistent with the graded expression pattern demonstrated by the mRNA profiles. The oncogene crk showed a graded mRNA as well as a graded protein expression pattern with survival in the lung adenocarcinomas, and was abundantly expressed in the lung tumor cells. These results strongly suggest that many survival-associated genes are expressed at the protein level and demonstrate similar mRNA and protein expression patterns.

TABLE 1
Top 100 Genes From Cross-Validation

| | | | P (Stage I vs. | | | |
|---|---|---|---|---|---|---|
| Gene | P (Normal vs. | % Change | Stage III T- | % Change | Coeficient | |
| Name | Tumor T-test) | in Tumor | test) | in Stage III | Beta | Unigene Comment |
| | | | | | | Apoptosis-related |
| BAG1 | 0.04 | −16% | 0.18 | 36% | 0.0023 | BCL2-associated athanogene |
| CASP4 | 0.56 | −6% | 0.02 | 57% | 0.0022 | caspase 4, apoptosis-related |
| | | | | | | cysteine protease |
| FADD | 1.62E−04 | 57% | 1.32E−03 | 49% | 0.0030 | Fas (TNFRSF6)-associated |
| | | | | | | via death domain |
| P63 | 9.73E−04 | 37% | 0.03 | 43% | 0.0010 | transmembrane protein (63 kD) |
| | | | | | | endoplasmic reticulum/Golgi |
| | | | | | | intermediate compartment |
| | | | | | | Cell adhesion and structure |
| 5T4 | 1.47E−08 | 139% | 0.05 | 43% | 0.0025 | 5T4 oncofetal trophoblast |
| | | | | | | glycoprotein |
| ITGA2 | 8.84E−05 | 109% | 0.96 | −1% | 0.0058 | integrin, alpha 2 (CD49B, |
| | | | | | | alpha 2 subunit of VLA-2 receptor) |
| KRT18 | 1.13E−18 | 192% | 0.26 | 18% | 0.0003 | keratin 18 |
| KRT19 | 2.57E−11 | 165% | 0.58 | 12% | 0.0003 | keratin 19 |
| KRT7 | 8.02E−08 | 126% | 0.11 | 55% | 0.0003 | keratin 7 |
| LAMB1 | 0.14 | −20% | 0.01 | 60% | 0.0027 | laminin, beta 1 |
| TMSB4X | 2.69E−05 | −33% | 0.01 | −18% | −0.0002 | thymosin, beta 4, X chromosome |
| TUBA1 | 0.01 | 65% | 0.04 | 39% | 0.0036 | tubulin, alpha 1 (testis specific) |
| | | | | | | Cell cycle and growth regulators |
| BMP2 | 0.54 | −21% | 0.27 | 47% | 0.0044 | bone morphogenetic protein 2 |
| CDC6 | 1.31E−05 | 1070% | 0.05 | 148% | 0.0124 | CDC6 (cell division cycle 6, S. |
| | | | | | | cerevisiae) |
| | | | | | | homolog |
| H2AFZ | 6.31E−04 | 31% | 2.12E−05 | 62% | 0.0008 | H2A histone family, member Z |
| PDAP1 | 2.52E−03 | 37% | 0.01 | 49% | 0.0056 | PDGFA associated protein 1 |
| POLD3 | 4.04E−06 | 127% | 0.16 | 28% | 0.0062 | polymerase (DNA directed), delta 3 |
| REG1A | 0.02 | 112% | 0.02 | −61% | 0.0004 | regenerating islet-derived 1 alpha |
| | | | | | | (pancreatic stone protein, |
| | | | | | | pancreatic thread protein) |
| S100P | 2.10E−08 | 1572% | 0.19 | 77% | 0.0001 | S100 calcium-binding protein P |
| SERPINE1 | 2.89E−03 | 72% | 0.25 | 30% | 0.0008 | serine (or cysteine) proteinase |
| | | | | | | inhibitor, |
| | | | | | | clade E (nexin) |
| STX1A | 8.65E−08 | 54% | 0.07 | 26% | 0.0031 | syntaxin 1A (brain) |
| | | | | | | Cell signaling |
| ADM | 0.05 | 39% | 0.04 | 117% | 0.0016 | adrenomedullin |
| AKAP12 | 8.53E−03 | −47% | 0.05 | 214% | 0.0010 | A kinase (PRKA) |
| | | | | | | anchor protein (gravin) 12 |
| ARHE | 0.06 | −39% | 0.05 | 87% | 0.0092 | ras homolog gene family, member E |
| DEFB1 | 0.11 | 41% | 0.29 | 79% | 0.0018 | defensin, beta 1 |
| GRB7 | 2.02E−03 | 38% | 0.63 | 15% | 0.0030 | growth factor receptor-bound |
| | | | | | | protein 7 |
| INHA | 0.04 | 23% | 0.13 | 30% | 0.0027 | inhibin, alpha |
| ITK | 0.05 | −68% | 0.18 | −188% | −0.0014 | IL2-inducible T-cell kinase |
| NACA | 0.02 | −9% | 0.23 | 8% | 0.0006 | nascent-polypeptide-associated |
| | | | | | | complex alpha polypeptide |
| STC1 | 0.03 | 150% | 0.01 | 188% | 0.0030 | stanniocalcin 1 |
| TNFAIP6 | 0.80 | −4% | 0.01 | 86% | 0.0052 | tumor necrosis factor, |
| | | | | | | alpha-induced protein 6 |
| VEGF | 6.50E−08 | 174% | 0.02 | 85% | 0.0013 | vascular endothelial growth factor |
| VLDLR | 0.02 | −41% | 0.10 | 110% | 0.0052 | very low density lipoprotein |
| | | | | | | receptor |
| WNT1 | 3.22E−04 | 252% | 0.98 | −1% | 0.0028 | wingless-type MMTV integration |
| | | | | | | site family, member 1 |
| WNT10B | 0.05 | 31% | 0.48 | 20% | 0.0022 | wingless-type MMTV integration |
| | | | | | | site family, member 10B |
| | | | | | | Chaperones |
| HSPA8 | 0.36 | 8% | 9.01E−04 | 51% | 0.0008 | heat shock 70 kD protein 8 |
| | | | | | | Receptors |
| ERBB2 | 0.04 | 92% | 0.37 | 120% | 0.0013 | v-erb-b2 avian erythroblastic |
| | | | | | | leukemia |
| | | | | | | viral oncogene homolog 2 |
| FXYD3 | 0.10 | 111% | 0.31 | 73% | 0.0046 | FXYD domain-containing |
| | | | | | | ion transport regulator 3 |
| HLA-B | 0.97 | 0% | 3.91E−04 | −40% | −0.0001 | major histocompatibility complex, |
| | | | | | | class I, B |
| HPCAL1 | 0.55 | 16% | 0.65 | 14% | 0.0052 | hippocalcin-like 1 |
| P2RX5 | 0.88 | 2% | 2.22E−03 | −28% | −0.0092 | purinergic receptor P2X, |
| | | | | | | ligand-gated ion channel, 5 |
| PEX7 | 3.11E−03 | 49% | 0.92 | 2% | 0.0172 | peroxisomal biogenesis factor 7 |
| SLC20A1 | 1.34E−03 | 58% | 0.02 | 66% | 0.0021 | solute carrier family 20 |
| | | | | | | (phosphate transporter), member 1 |
| SLC2A1 | 3.21E−18 | 246% | 0.04 | 24% | 0.0030 | solute carrier family 2 |
| | | | | | | (facilitated glucose transporter), |
| | | | | | | member 1 |
| VDAC2 | 3.15E−05 | 33% | 0.12 | 31% | 0.0006 | voltage-dependent anion channel 2 |
| | | | | | | Enzymes, cellular metabolism |
| ALDH8 | 1.47E−05 | 150% | 0.49 | 20% | 0.0032 | aldehyde dehydrogenase 8 |
| ALDOA | 8.60E−06 | 48% | 0.09 | 22% | 0.0002 | aldolase A, fructose-bisphosphate |
| ATP2B1 | 1.02E−04 | −42% | 0.03 | 73% | 0.0036 | ATPase, Ca++ transporting, |
| | | | | | | plasma membrane 1 |
| CDS1 | 0.84 | 3% | 0.06 | 68% | 0.0057 | CDP-diacylglycerol synthase |
| | | | | | | (phosphatidate |
| | | | | | | cytidylyltransferase) 1 |
| CSTB | 1.57E−04 | 50% | 0.15 | 34% | 0.0001 | cystatin B (stefin B) |
| CTSL | 0.48 | −10% | 0.03 | 67% | 0.0007 | cathepsin L |
| CYP24 | 3.16E−06 | N/A | 0.97 | 2% | 0.0008 | cytochrome P450, subfamily |
| | | | | | | XXIV |
| | | | | | | (vitamin D 24-hydroxylase) |
| FUCA1 | 1.40E−01 | 22% | 3.04E−06 | −35% | −0.0013 | fucosidase, alpha-L-1, tissue |
| FUT3 | 1.07E−07 | 114% | 0.97 | −1% | 0.0033 | fucosyltransferase 3 (galactoside |
| | | | | | | 3(4)-L- |
| | | | | | | fucosyltransferase, |
| | | | | | | Lewis blood group included) |
| GAPD | 1.00E−18 | 173% | 8.38E−04 | 46% | 0.0002 | glyceraldehyde-3-phosphate |
| | | | | | | dehydrogenase |
| GCNT1 | 0.01 | 86% | 0.02 | 132% | 0.0081 | glucosaminyl (N-acetyl) |
| | | | | | | transferase 1, |
| | | | | | | core 2 (beta-1,6-N- |
| | | | | | | acetylglucosaminyltransferase) |
| HMBS | 3.94E−05 | 56% | 0.25 | 17% | 0.0070 | hydroxymethylbilane synthase |
| KYNU | 0.03 | 62% | 0.06 | 124% | 0.0007 | kynureninase (L-kynurenine |
| | | | | | | hydrolase) |
| MLN64 | 0.20 | 32% | 0.42 | 80% | 0.0007 | steroidogenic acute regulatory |
| | | | | | | protein related |
| MSH3 | 0.35 | 12% | 0.02 | 41% | 0.0140 | mutS (E. coli) homolog 3 |
| MT2A | 0.01 | −45% | 0.03 | 79% | 0.0002 | metallothionein 2A |
| NME2 | 8.90E−13 | 58% | 0.01 | 22% | 0.0004 | non-metastatic cells 2, protein |
| | | | | | | (NM23B) expressed in |
| NP | 0.15 | 14% | 0.01 | 32% | 0.0010 | nucleoside phosphorylase |
| PACE | 1.21E−04 | 69% | 0.11 | 64% | 0.0009 | paired basic amino acid cleaving |
| | | | | | | enzyme |
| | | | | | | (furin, membrane associated |
| | | | | | | receptor protein) |
| PDE7A | 0.12 | 33% | 0.01 | −35% | −0.0187 | phosphodiesterase 7A |
| PLGL | 0.04 | −68% | 0.35 | −170% | −0.0011 | plasminogen-like |
| PPIF | 8.84E−07 | 125% | 0.02 | 57% | 0.0042 | peptidylprolyl isomerase F |
| | | | | | | (cyclophilin F) |
| PTPRCAP | 6.65E−01 | −6% | 0.02 | −27% | −0.0029 | protein tyrosine phosphatase, |
| | | | | | | receptor type, |
| | | | | | | C-associated protein |
| RPC | 6.47E−03 | 33% | 0.05 | 43% | 0.0070 | RNA 3′-terminal phosphate cyclase |
| SC4MOL | 0.08 | −38% | 0.02 | 58% | 0.0069 | sterol-C4-methyl oxidase-like |
| SLC1A6 | 0.07 | −32% | 0.12 | 86% | 0.0069 | solute carrier family 1 (high |
| | | | | | | affinity |
| | | | | | | aspartate/glutamate transporter), |
| | | | | | | member 6 |
| UBC | 0.02 | −17% | 0.05 | 14% | 0.0007 | ubiquitin C |
| UGP2 | 0.12 | 13% | 1.43E−05 | 59% | 0.0056 | UDP-glucose pyrophosphorylase 2 |
| UQCRC2 | 0.63 | −3% | 0.26 | 17% | 0.0030 | ubiquinol-cytochrome c reductase |
| | | | | | | core protein II |
| | | | | | | Transcription and translation |
| COPEB | 0.10 | −33% | 0.26 | 25% | 0.0016 | core promoter element binding |
| | | | | | | protein |
| CRK | 0.10 | 32% | 0.03 | 48% | 0.0098 | v-crk avian sarcoma virus CT10 |
| | | | | | | oncogene homolog |
| DBP | 0.61 | 4% | 3.27E−03 | −23% | −0.0048 | D site of albumin promoter |
| | | | | | | (albumin D-box) binding protein |
| GARS | 1.55E−04 | 38% | 0.02 | 48% | 0.0012 | glycyl-tRNA synthetase |
| HRB | 3.74E−03 | 25% | 2.88E−03 | 45% | 0.0092 | HIV-1 Rev binding protein |
| HSU53209 | 0.34 | 6% | 0.07 | −14% | −0.0034 | transformer-2 alpha (htra-2 alpha) |
| PRDM2 | 3.08E−05 | −50% | 0.02 | −30% | −0.0106 | PR domain containing 2, with ZNF |
| | | | | | | domain |
| RELA | 0.26 | −7% | 0.01 | 20% | 0.0034 | v-rel avian reticuloendotheliosis |
| | | | | | | viral oncogene homolog A |
| RPS26 | 0.55 | 12% | 0.13 | 37% | 0.0001 | ribosomal protein S26 |
| RPS3 | 9.97E−03 | 12% | 0.24 | 15% | 0.0002 | ribosomal protein S3 |
| RPS6KB1 | 0.83 | 3% | 0.02 | 37% | 0.0112 | ribosomal protein S6 kinase, 70 kD, |
| | | | | | | polypeptide 1 |
| SUI1 | 0.34 | 5% | 0.05 | 18% | 0.0005 | putative translation initiation factor |
| TIEG | 0.05 | −24% | 0.15 | 16% | 0.0073 | TGFB inducible early growth |
| | | | | | | response |
| TMF1 | 4.64E−04 | 51% | 0.10 | 27% | 0.0198 | TATA element modulatory factor 1 |
| | | | | | | Unknown function |
| B1 | 0.75 | −2% | 1.75E−03 | −21% | −0.0074 | PTH-responsive osteosarcoma B1 |
| | | | | | | protein |
| FEZ2 | 0.41 | 9% | 0.27 | 20% | 0.0066 | fasciculation and elongation |
| | | | | | | protein zeta 2 (zygin II) |
| HPIP | 3.31E−04 | 31% | 0.05 | −19% | −0.0059 | hematopoietic PBX-interacting |
| | | | | | | protein |
| KIAA0005 | 2.21E−04 | 40% | 0.02 | 45% | 0.0010 | KIAA0005 gene product |
| KIAA0020 | 0.01 | 35% | 4.91E−03 | 73% | 0.0072 | KIAA0020 gene product |
| KIAA0084 | 1.93E−05 | −26% | 4.79E−04 | −27% | −0.0024 | KIAA0084 protein |
| KIAA0153 | 1.90E−03 | 40% | 0.14 | 16% | 0.0027 | KIAA0153 protein |
| KIAA0263 | 4.96E−03 | −25% | 1.87E−05 | −28% | −0.0044 | KIAA0263 gene product |
| KIAA0317 | 3.35E−06 | 32% | 0.07 | 17% | 0.0055 | KIAA0317 gene product |
| MGB1 | 0.27 | 125% | 0.33 | 459% | 0.0018 | mammaglobin 1 |
| NULL | 2.98E−04 | 26% | 0.76 | 4% | 0.0037 | Homo sapiens pTM5 mariner-like |
| | | | | | | transposon mRNA, partial |
| | | | | | | sequence |
| NULL | 0.68 | −3% | 2.66E−03 | −30% | −0.0005 | Human unproductively rearranged |
| | | | | | | Ig mu-chain mRNA V-region |
| | | | | | | (VD), 5′ end, clone mu-3A1A |

Bolded genes were also significant for survival in 43 tumor training set (FIG. 4B)

Table 2. The primer sequences flanking genomic fragments for the genes selected for gene amplification analysis. The PACE4 gene was used as a control since its chromosomal location at 15q26 is very close to PACE (15q25) and would detect increased copy number of the chromosome arm.

TABLE 2
Primer Sequences

| | | Chromosome | | |
|---|---|---|---|---|
| Gene Symbol | GenBank ID | Location | Primer sequence (5′-3′) | |
| REG1A | M18963 | 2p12 | TCC AAA GAC TGG GGT AGG T | (SEQ ID NO:4) |
| | | | AGC AAT TAC AAC GGA GTC AA | (SEQ ID NO:5) |
| KYNU | U57721 | 2q21-22 | TTT TAG AGA ACA ACT TGT AAT GGA GCC | (SEQ ID NO:6) |
| | | | AGT GCC TCA GCT TAT CTT CCT CA | (SEQ ID NO:7) |
| AKAP12 | M96322 | 6q24-25 | ACG GAG TGT GCC AAA ACT AAA AA | (SEQ ID NO:8) |
| | | | CGG TCC AAA CAA CCA TAA CAA GTA | (SEQ ID NO:9) |
| MGB1 | U33147 | 11q13 | AGT GAA ACT TTG AGC TCT CGT CTA ACC | (SEQ ID NO:10) |
| | | | CAG GGA AGG GTG ATG GAT TTG TTT | (SEQ ID NO:11) |
| WNT1 | X03072 | 12q13 | TGC GTT TTC TCC GGG TCC TCC TAA | (SEQ ID NO:12) |
| | | | CCC CCA ACC TCA TTC CCA CAT CAT | (SEQ ID NO:13) |
| KRT18 | M26326 | 12q13 | CCT CCC TAC CTC CAT CCT TCT CAC | (SEQ ID NO:14) |
| | | | CAG TGC CCC TCC CCT CTT TTC | (SEQ ID NO:15) |
| PACE | X17094 | 15q25-26 | CAC CCT CTA GTT GAA CCC CCT TAT | (SEQ ID NO:16) |
| | | | ACC TGC CTA CCC TCC CTC TG | (SEQ ID NO:17) |
| PACE4 | M80482 | 15q26 | CTT GCT TCA TTG ATT TCT CTT TCA | (SEQ ID NO:18) |
| | | | CTT TCT CCA TAT TGT CCT GCT CC | (SEQ ID NO:19) |
| ERBB2 | M11730 | 17q21 | AAC AAA AGC GAC CCA TTC AGA G | (SEQ ID NO:20) |
| | | | CTC CCC TGG GTC TTT ATT TCG TC | (SEQ ID NO:21) |
| SLC1A6 | U18244 | 19p13 | CGG CCG TCA TCG AGC ACT TG | (SEQ ID NO:22) |
| | | | GAG GCC CCT CAC ATA GCA CTC TC | (SEQ ID NO:23) |
| CYP24 | L13286 | 20q13 | CGA CTA CCG CAA AGA AGG CTA C | (SEQ ID NO:24) |
| | | | CCA ACC CCA GGG AAC TCT AAC T | (SEQ ID NO:25) |

### Example 2

Proteomic Analysis of Lung Adenocarcinomas

A. Methods

Tissue Specimens and Preparation

All lung tumors and adjacent normal lung tissue were obtained at the time of surgery at the University of Michigan Hospital from May 1991 to July 2000. Consent was received from all patients and the project approved by the Institutional Review Board. Patients' medical records were reviewed and patient identifiers coded to protect confidentiality. The samples were transported to the laboratory in Dulbecco's modified Eagle medium (Life Technologies, Gaithersburg Pa.) on ice. Sixty-four stage I lung adenocarcinomas, 29 stage III lung adenocarcinomas and 10 uninvolved lung tissue samples were examined. A portion of each sample was embedded in OCT (Miles Scientific, Naperville, Ill.), frozen in isopentane cooled with liquid nitrogen for cryostat sectioning and stored at −80° C. Hematoxylin-stained cryostat sections (5 μm), prepared from tumor pieces to be utilized for protein or mRNA isolation, were evaluated by a study pathologist (TJG), as well as compared to H&E stained sections made from paraffin blocks of the same tumors. The same selected region of the tumor was used for protein and mRNA isolation. Specimens were excluded if there was: (1) unclear or mixed histology (e.g., adenosquamous); (2) tumor cellularity less than 70%; (3) potential metastatic origin as indicated by previous tumor history; (4) extensive lymphocytic infiltration or fibrosis; or (5) the patient had experienced chemotherapy or radiotherapy. Tumors were histopathologically divided into two categories: bronchial-derived, if they exhibited invasive features with architectural destruction, or bronchioloalveolar, if they exhibited preservation of the lung architecture.

2D-PAGE and Protein Quantification

Analytical 2D-PAGE was performed as described previously (Strahler, J. R., Kuick, R., and Hanash, S. M. In: Creighton, T. (ed.), Protein Structure: A Practical Approach, pp. 65-92. Oxford: IRL, 1989). After separation, the protein spots were visualized by a photochemical silver-based staining technique (Merril, C. R., Dunau, M. L., and Goldman, D. A rapid sensitive silver stain for polypeptides in polyacrylamide gels. Anal. Biochem., 101: 201-207, 1981). Each gel was scanned using a Kodak CCD camera. Spot detection was accomplished by employing Bio Image Visage System software (Bioimage Corp., Ann Arbor, Mich.). Each gel generated 1600-2200 detectable spots, of which 820 spots were selected for quantitative measurement. The integrated intensity, which is the value of each spot, was calculated as the measured optical density units multiplied by square millimeters. The protein spots from each gel were matched to the 820 spots on a “master” gel (Kuick, R. D., Skolnick, M. M., Hanash, S. M., and Neel, J. V. A two-dimensional electrophoresis-related laboratory information processing system: spot matching. Electrophoresis, 12: 736-746, 1991) to allow identification of identical polypeptides between each gel. A total of 250 spots were chosen as ubiquitously expressed reference spots to allow adjustment for subtle variation in protein loading and gel staining. Each of the 820 spots was then mathematically adjusted in relation to the reference spots (Kuick, R. D., Hanash, S. M., Chu, E. H. Y., and Strahler, J. R. A comparison of some adjustment techniques for use with quantitative spot data from two-dimensional gels. Electrophoresis, 8: 199-204, 1987). The resulting data can be accessed with common spreadsheet software.

Mass Spectrometry and Polypeptide Sequencing. Some of the polypeptides included in the analysis had been identified prior to this study on the basis of sequencing (Hanash, S. M., Strahler, J. R., Chan, Y., Kuick, R., Teichroew, D., Neel, J. V., Hailat, N., Keim, D. R., Gratiot-Deans, J., Ungar, D., and Richardson, B. C. Data base analysis of protein expression patterns during T-cell ontogeny and activation. Proc. Natl. Acad. Sci. USA, 90: 3314-3318, 1993). For MALDI-MS, the protein spots identified for analysis were cut from preparative 2D gels using extracts from A549 lung adenocarcinoma cells (ATCC, Rockville, Md.). The run parameters were the same as those used for the analytical 2D gels. Identification of proteins was performed by trypsin digestion followed by MALDI-MS. This allowed for a “fingerprint” to be created for each protein spot based on the molecular weight of the trypsin-digested products. The masses were compared to known trypsin digest databases using the MS-FIT database (University of California at San Francisco). The results were given as probability matches to known tryptic digest patterns established by multiple databases. Affymetrix Oligonucleotide Microarrays. Total RNA was isolated from 76 of the tumors and 9 of the normal lung samples using Trisol reagent (Life Technologies, Gaithersburg, Pa.). The resulting RNA was subjected to further purification using RNeasy spin columns (Qiagen Inc., Valencia, Calif.) and used to generate cRNA probes. All protocols used for mRNA reverse transciptase, second strand synthesis, production of cDNA and cRNA amplification, hybridization and washing conditions for the 6800 gene HuGeneFL oligonucleotide arrays were as provided by the manufacturer (Affymetrix, Santa Clara, Calif.). The arrays were scanned using a GeneArray scanner with data analysis performed using GeneChip 4.0 software (Affymetrix). Details of data trimming and normalization are described elsewhere (Giordano, T. J., Shedden, K. A., Schwartz, D. R., Kuick, R., Taylor, J. M. G., Lee N., Misek, D. E., Greenson, J. K., Kardia, S. L. R., Beer, D. G., Rennert, G., Cho, K. R., Gruber, S. B., Fearon, E. R., and Hanash, S. Organ-specific molecular classification of lung, colon and ovarian adenocarcinomas using gene expression profiles. Am. J. Pathol. 159:1231-1238, 2001).

K-ras Mutational Status

Genomic DNA was isolated from each tumor sample and fifty ng was subjected to PCR amplification using the primers that encompass codons 12 and 13 of the K-ras gene. The sequences of forward and reverse primers are 5′ TATAAGGCCTGCTGAAAAT 3′ (SEQ ID NO:26) and 5′ CCTGCACCAGTAATATGC 3′ (SEQ ID NO:27), respectively. Two ng of purified PCR products containing the exon 1 of the K-ras gene were then subjected to thermal cycle sequencing with an internal nested primer (5′ AGGCCTGCTGAAAATGACT 3′; SEQ ID NO:28) and resolved in 8% urea PAGE gels, dried, and exposed to Phosphor-Image screens and visualized using a Phosphor-Image scanner (Molecular Dynamics, Sunnyvale, Calif.). The mutations were determined by comparing each tumor DNA sequences of K-ras 12th and 13th codon to its wild type sequence GGTGGC.

2D Western Blotting

Protein extracts of A549 lung adenocarcinoma cells were run on 2D gels using the identical conditions as used for the analytical 2D gels. The separated proteins were transferred onto polyvinylidene fluoride membranes and incubated for 2 h at room temperature with a blocking buffer consisting of TBST (Tris-buffered saline, 0.01% Tween 20) and 5% nonfat dry milk. Individual membranes were washed and incubated with anti-Erp57 (GRP58) mouse monoclonal antibody (SPA-725, 0.6 μg/mL, StressGen Biotechnologies Corp, BC Canada) and anti-UCHL1 (PGP9.5) rabbit polyclonal antibody (0.6 μg/mL, Biogenesis, Kingston, N.H.) for 1 h at room temperature. After additional washes with TBST, the membranes were incubated with a secondary antibody conjugated with horseradish peroxidase (HRP) at a 1:5000 dilution for 1 h, further washed and then incubated 1 min with ECL (enhanced chemiluminescence) (Pierce, Ill.)

Immunohistochemistry of Tissue Microarrays

A tissue microarray block was constructed according to the method of Kononen (Kononen et al., Nat. Med., 4: 844 [1998]) and utilized the best representative morphological areas of the tumors in this study. Deparaffinized sections of the pulmonary adenocarcinoma tissue microarray were microwaved after pretreatment in citric acid to retrieve antigenicity. The sections were incubated with blocking solution containing phosphate-buffered saline and 1% bovine serum album for 60 min at room temperature. The antibodies examined included the anti-UCHL1 (PGP9.5) rabbit polyclonal antibody, anti-Erp57 (GRP58) mouse monoclonal antibody (used for frozen tissue sections) and anti-p53 mouse monoclonal antibody (1.0 μg/mL, Dako Corporation, Carpinteria, Calif.). The sections were incubated with primary antibodies overnight at 4° C. The immuno-complex was visualized by the immunoglobulin enzyme bridge technique using a Vector ABC-peroxidase kit (Vector Laboratories, Burlingame, Calif.) with 3,3′ diaminobenzidine tetrachloride as a substrate. The sections were lightly counter-stained with hematoxylin.

mRNA in situ Hybridization of Tissue Microarrays

Because of the lack of availability of an antibody, the presence and cellular abundance of mRNA for TPI was determined using in situ hybridization (ISH) with the tissue microarrays. Biotinylated oligonucleotides (*GCCCCATTAGTCACTTTGTAGC; SEQ ID NO:29) and (*CAGAGGGACTCG GAGTAATCG; SEQ ID NO:30) were synthesized using the published TPI mRNA sequence (Brown et al., Mol. Cell Biol., 5:1694 [1985]. ISH was carried out as previously described (Frantz et al., J. Pathol., 195:87 [2001]). The sections were deparaffinized, hydrated and washed with PBS for 10 min. Next, they were treated with proteinase K (20 μg/mL in PBS) for 10 min at 37° C., followed by treatment with 0.2 M HCl. Postfixation was performed using 4% (w/v) paraformaldehyde. The sections were then hybridized overnight at 42° C. with the labeled oligonucleotides. After hybridization, the sections were washed using increased stringency with SSC (0.15 M NaCl, 15 mM Na citrate, pH 7.2) and then subjected to immunostaining with a mouse anti-biotin monoclonal antibody. After amplification of the biotin-antibody complex with biotin-labeled horse anti-mouse IgG and alkaline phosphatase-labeled streptavidin, the sites of alkaline phosphatase were visualized with NBT/BCIP as previously reported (Frantz et al., J. Pathol., 195: 87 [2001]). For each run, hybridization with an anti-sense probe and without the anti-sense probe (control) was used. ISH of beta-actin (a gift from Dr. Sakiyama, Chiba, Japan) was performed on tissue microarray sections of all the lung tissue specimens to assess their mRNA integrity. The sections were lightly counterstained with nuclear fast red.

Statistical Analysis

S-plus software (Insightful Corp, Seattle, Wash.) was used to investigate the specific features of protein expression. T-tests were used to identify the differences in mean values between comparison groups. The relationship between the levels of protein and mRNA expression was examined using the Spearman correlation coefficient statistical method (Sadahiro et al., Cancer, 92:1251 [2001]). A P-level of <0.05 (two-sided) was considered statistically significant.

B. Results

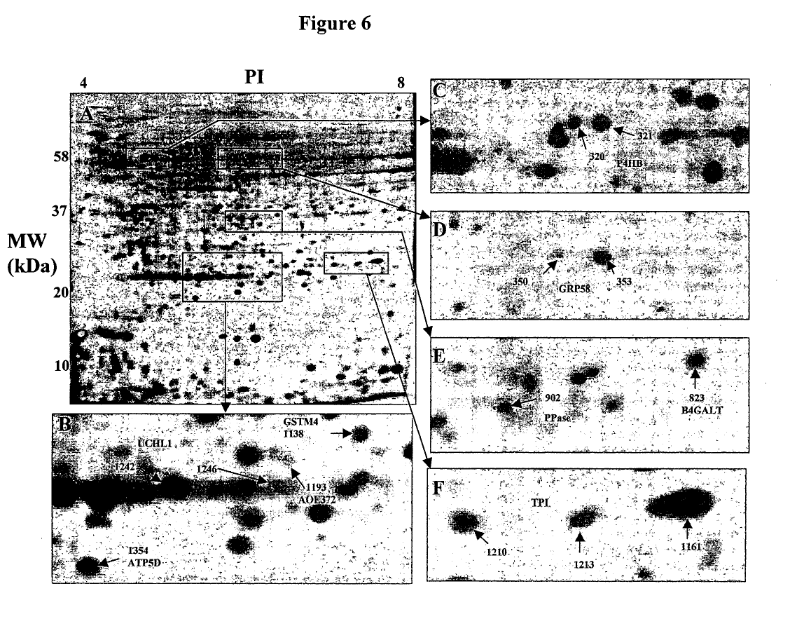

By comparing protein expression levels between 93 lung adenocarcinomas and 10 uninvolved lung samples, 9 different enzyme proteins were identified using 2D-PAGE and MALDI-MS or peptide sequencing. These proteins were all significantly increased in the lung adenocarcinomas (a 1.4- to 10.6-fold increase). They included the antioxidant enzyme AOE372, ATP synthase subunit d (ATP5D), beta 1,4-galactosyltransferase (B4GALT), cytosolic inorganic pyrophosphatase (PPase), glucose regulated 58 kDa protein (GRP58), glutathione-s-transferase M4 (GSTM4), prolyl 4-hydroxylase, beta subunit (P4HB), triosephosphate isomerase (TPI) and ubiquitin thiolesterase (UCHL1) (FIG. 6 and Table 3). Some of these proteins were identified as having multiple isoforms. The proteins in general exhibited increased expression of all individual isoforms relative to normal lung, except for P4HB, which demonstrated one isoform that was significantly overexpressed in lung adenocarcinomas, and one isoform that was unchanged relative to uninvolved lung tissue.

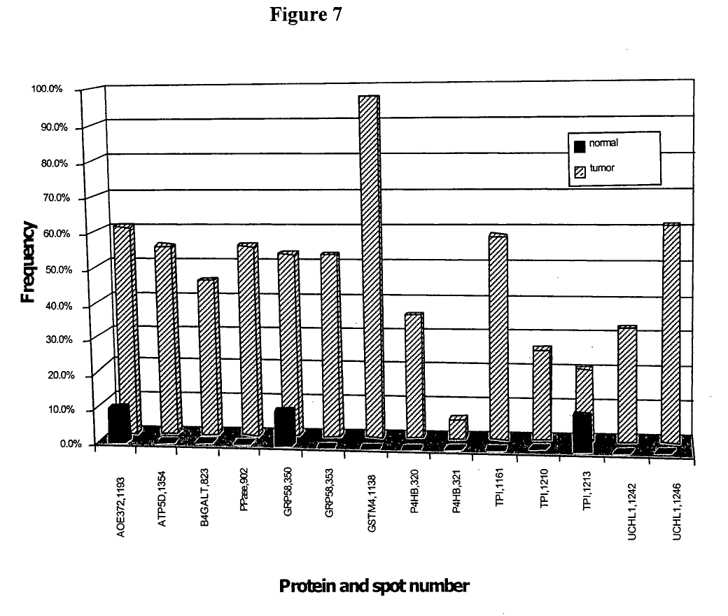

The frequency of protein expression in the individual tumor and normal samples was determined using the value of the normal lung, with the mean+2SD as the cutoff value. Proteins that were significantly increased in lung adenocarcinomas were detected at this level in 35.5% to 96.8% of the tumor samples (FIG. 7). Only three proteins (or one isoform) were detected at this level in 10% or less of the normal lung samples.

The protein expression values of the nine tumor-associated enzyme proteins were examined for potential correlation with clinical-pathological variables including: tumor stage, tumor classification, tumor differentiation, angiolymphatic invasion, lymphocytic response, P53 nuclear protein accumulation, K-ras 12^{th}/13^{th} codon mutation, smoking status (Table 4). AOE372 was found to be overexpressed in poorly differentiated tumors relative to moderately differentiated tumors (P=0.04). PPase was increased in bronchial-derived adenocarcinomas (P=0.02) and in patients with a positive smoking history (P=0.04). GRP58 (isoform #353) was increased in tumors with K-ras mutations (P=0.04). P4HB (isoform #320) was decreased in tumors having a positive lymphocytic response (P=0.03). TPI (isoform #1213) was increased in poor relative to well-differentiated tumors (P=0.04) and was decreased in tumors from patients with a smoking history (P=0.04). UCHL1 (isoform #1242) in contrast, was over-expressed in patients with a smoking history (P=0.01). ATP5D and B4GALT were not correlated to any of the clinical-pathological variables. No relationship was observed between the individual protein isoforms and the presence of p53 nuclear staining or angiolymphatic invasion.

To examine whether the changes in protein expression may be due to transcriptional or other mechanisms of regulation, a comparison of the mRNA expression values and the protein expression values within the same tumor samples was made. Table 4 shows the correlation coefficients for the proteins for which probes for the corresponding genes were also present on the oligonucleotide arrays. Both GRP58 isoforms were significantly correlated with their respective mRNA levels in these tumors (P<0.05). This suggests that the increase in GRP58 protein expression in these tumors is associated with a corresponding increase in its mRNA, thus reflecting transcriptional regulation. No statistically significant correlation of the other protein isoforms with their respective mRNA expression was observed. The relative mRNA expression of these genes between lung adenocarcinomas and uninvolved normal lung tissues is shown in Table 5. The levels of mRNA for AOE372, GRP58, P4HB, TPI and UCHL1 were found to be significantly increased in lung adenocarcinomas relative to normal lung (P<0.005), however ATP5D, B4GALT, PPase and GSTM4 were not.

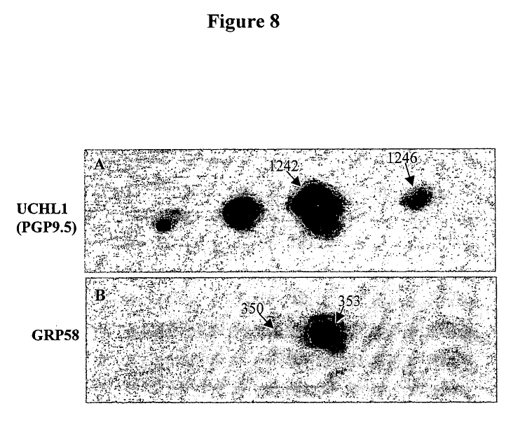

2D Western blot analysis of A549 lung adenocarcinoma cell lines using an antibody to UCHL1 (PGP9.5) revealed four immunoreactive protein spots (FIG. 8A). Two of the spots (#1242 and #1246) were also identified using MALDI-MS. The other two spots were not quantified in the primary lung tumors because most of the gels demonstrated multiple overlapping patterns in that region. Spot #1246 was the predominant isoform identified in the lung tumors, and a high level of expression of UCHL1 was present in 61.3% of these tumors (Table 3 and FIG. 7). The immunohistochemical analysis of UCHL1 in tissue microarrays using the same antibody demonstrated abundant cytoplasmic staining in the lung tumor cells and very low levels in normal lung tissue.

2D Western blot analysis of GRP58 confirmed the two spots (#353 and #350) identified by peptide sequencing from 2D gels (FIG. 8B). The level of GRP58 isoform (#353) was found to be 3.2-fold higher than the other isoform (#350), however both demonstrated the same frequency (52.7%) of expression in the tumor samples (Table 3 and FIG. 7). Immunohistochemical analysis of GRP58 was performed on frozen lung adenocarcinoma tissue samples due to the lack of antibody reactivity using formalin-fixed tissue. GRP58 staining was abundant within the cytoplasm of the tumor cells with lower levels of staining detected in normal lung from the same patients.

TPI mRNA analysis of tumor arrays using ISH indicated higher level of mRNA expression in the cytoplasm of the tumor cells as compared with normal lung tissue (FIGS. 4E and F). This finding is consistent with the increased TPI mRNA in lung tumors relative to normal lung tissue determined using the oligonucleotide arrays (Table 3, P<0.0001).

### Example 3

Discordant Protein and mRNA Expression in Lung Adenocarcinomas

A. Methods

Tissues

Fifty-seven stage I, 19 stage III lung adenocarcinomas, and 9 non-neoplastic lung tissue samples were used for protein and mRNA analyses. Patient consent was obtained and the project approved by the Institutional Review Board. All tissues were obtained after resection at The University of Michigan Health System between May 1991 and July 1998. Tissues were all snap frozen in liquid nitrogen and then stored at −80° C. The patients included 46 females and 30 males ranging in age from 40.9 to 84.6 (average 63.8) years. Most patients (66/76) demonstrated a positive smoking history. Sixty-one tumor samples were classified as bronchial-derived (BD), 14 as bronchoalveolar (BA), and one had both features (BA/BD). Eighteen tumor samples were classified as well-differentiated, 38 as moderate, and 19 as poorly differentiated adenocarcinomas. Hematoxylin-stained cryostat sections (5 m), prepared from the same tumor pieces to be utilized for protein and mRNA isolation, were evaluated by a study pathologist and compared to H&E sections made from paraffin blocks of the same tumors. Specimens were excluded from analysis if they showed unclear or mixed histology (e.g., adenosquamous), tumor cellularity less than 70%, potential metastatic origin as indicated by previous tumor history, extensive lymphocytic infiltration, fibrosis, or if the patient had received prior chemotherapy or radiotherapy.

Oligonucleotide Array Hybridization

The HuGeneFL oligonucleotide arrays (Affymetrix, Santa Clara, Calif.), containing 6800 genes, were used in this study. Total RNA was isolated from all samples using Trisol reagent (Life Technologies, Gaithersburg, Pa.). The resulting RNA was then subjected to further purification using RNeasy spin columns (Qiagen Inc., Valencia, Calif.). Preparation of cRNA, hybridization and scanning of the HuGeneFL Arrays were performed according to the manufacturer's protocol (Affymetrix, Santa Clara, Calif.). Data analysis was performed using GeneChip 4.0 software. Each tumor's gene expression profile was normalized to the median gene expression profile for the entire sample. Details of data trimming and normalization are described elsewhere (Giordano et al., Am. J. Pathol. 159:1231 [2001]).

2D-PAGE and Quantitative Protein Analysis

Tissue for both protein and mRNA isolation came from contiguous areas of each sample. Protein separation using 2D PAGE, silver staining, and digitization were performed as previously described (Strahler et al., (1989) In Protein structure: A practical approach, ed. Creighton, T. (IRL, Oxford), pp. 65-92; Merril et al., Anal. Biochem. 101, 201 [1981]). Spot detection and quantification were accomplished utilizing Bio Image Visage System software (Bioimage Corp., Ann Arbor, Mich.). The integrated intensity of each spot was calculated as the measured optical density units×square millimeters. Of the total possible 2000 spots detectable on each gel, 820 spots on each sample's gel were matched using a Gel-ed match program with the same spots on a chosen “master” gel. In each sample, 250 ubiquitously expressed reference spots were used to adjust for variations between gels, such as that created by subtle differences in protein loading or gel staining. Slight differences due to batch were corrected after spot size quantification.

Mass Spectrometry and 2D Western Blotting

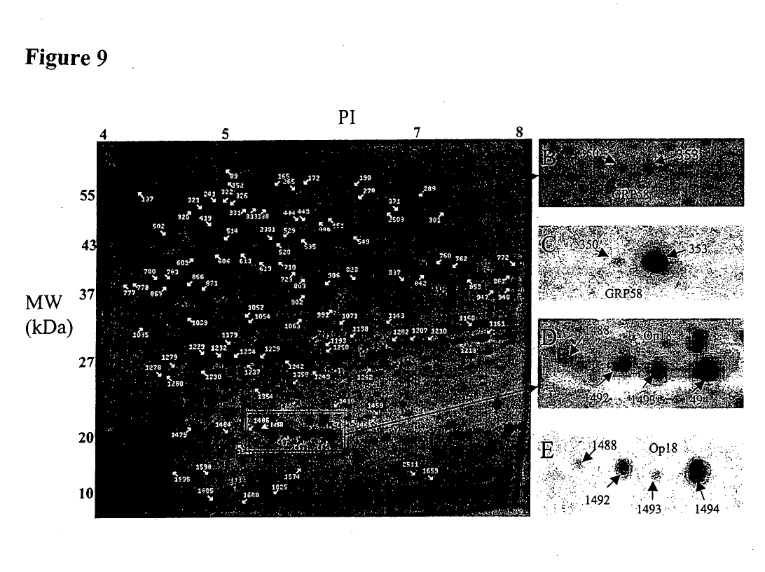

Preparative 2D gels were run using extracts from A549 lung adenocarcinoma cells (obtained from ATCC) and the identical experimental conditions as the analytical 2D gels, except a 30% greater protein loading. The resolved protein gels were silver-stained using successive incubations in 0.02% sodium thiosulfate for 2 min, 0.1% silver nitrate for 40 min, and 0.014% formaldehyde plus 2% sodium carbonate for 10 minutes. For protein identification, protein polypeptides underwent trypsin digestion followed by matrix-assisted laser desorption/ionization mass spectrometry (MALDI-MS) using a MALDI-TOF Voyager-DE Mass Spectrometer (Perseptive Biosystems, Framingham, Mass.). The masses were compared to known trypsin digest databases using the MS-FIT database (University of California at San Francisco). Some of the polypeptides included in the analysis had been identified prior to this study on the basis of sequencing (Hanash et al., PNAS 90:3314 [1993]). The identified protein spots are shown in FIG. 9A. The method for 2D PAGE western blot verification was as described previously (Brichory et al., PNAS 98:9824 [2001]). The 2D Western blots of the GRP58 and Op18 are shown in FIGS. 9C and E, the others such as GRP78, GRP75, HSP70, HSC70, KRT8, KRT18, KRT19, Vimentin, ApoJ, 14-3-3, Annexin I, Annexin II, PGP9.5, DJ-1, GST-pi, and PGAM are described in the above examples.

Statistical Analysis

Missing values were replaced with the protein spot's mean value. The transform x→log (1+x) was applied to normalize all protein expression values. The relationship between protein and mRNA expression levels within the same samples was examined using the Spearman correlation coefficient analysis (Lavens-Phillips et al., Am. J. Respir. Cell Mol. Biol. 23:566 [2000]). To identify potentially significant correlations between gene and protein expression, an analytical strategy similar to SAM (Significance Analysis of Microarrays) (Tusher et al., PNAS 98:5116 [2001]), which uses a permutation technique to determine the significance of changes in gene expression between different biological states, was utilized. To obtain permuted correlation coefficients between gene and protein expression, genes were first exchanged in such a way that permuted correlation coefficients were calculated based on pseudo pairs of genes and proteins. The distribution of permuted correlation coefficients became stable after 60 permutations. This procedure was then repeated sixty times to obtain sixty sets of permuted correlation coefficients. For each of the sixty permutations, the correlations of genes and proteins were ranked such that ρₚ (i) denotes the i th largest correlation coefficient for p th permutation. Hence, the expected correlation coefficient, ρ_{E} (i), was the average over the sixty permutations,

$${ρ}_{E} ( i ) = {\sum }_{p = 1}^{60} {ρ}_{p} ( i ) / 60.$$

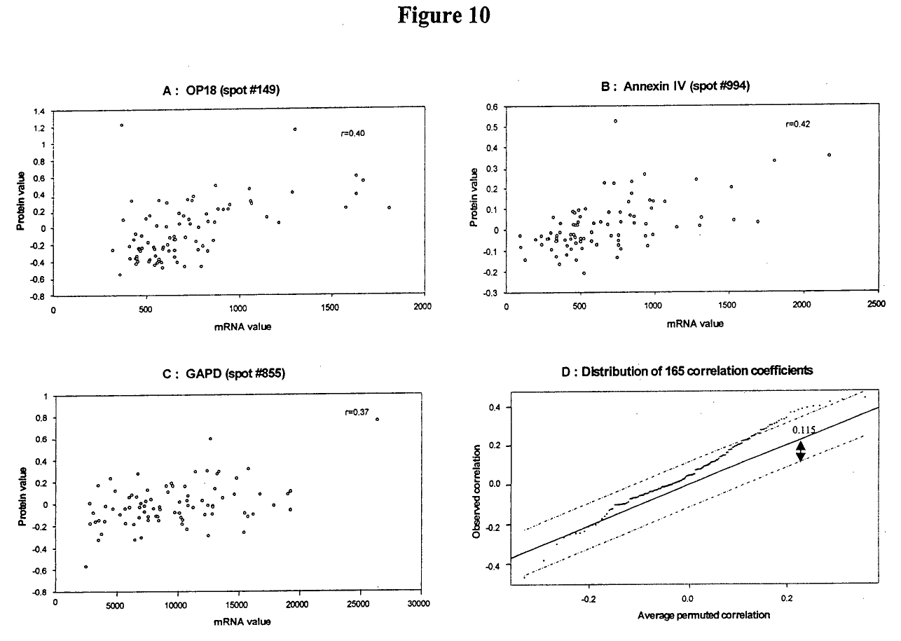

A scatter plot of observed correlations (ρ(i)) vs. the expected correlations is shown in FIG. 10D. For this study, a threshold Δ=0.115 was used so that correlation would be considered significant if the absolute value of difference between ρ(i) and ρ_{E} (i) was greater than the threshold. Twenty nine (including one with observed correlation coefficient −0.4672) out of 165 pairs of gene and protein expression were called significant in such criteria, and the permuted data generated an average of 5.1 falsely significant pairs of gene and protein expression. This provided an estimated FDR (false discovery rate, the percentage of pairs of gene and protein expression identified by chance) for the data set.

B. Results

Correlation of Individual Proteins and mRNA Expression within Each Tumor

165 protein spots on 2D gels representing 98 genes were used for a comparison of protein levels with mRNA levels for a cohort of 85 lung adenocarcinomas and normal lung samples were examined. Of the 165 protein spots, 69 proteins were represented by only one known spot on 2D gels for an individual gene, whereas 96 protein spots showed multiple protein products from 29 different genes. 2D Western blotting verified the proteins identified by mass spectrometry when specific antibodies were available. Spearman correlation coefficients of the proteins and their associated mRNA for each protein spot were generated using all 76 lung adenocarcinomas and 9 non-neoplastic lung tissues (Tables 6 and 7; FIGS. 9 and 10). The correlation coefficients (r) ranged from 0.467 to 0.442 (FIG. 10D). A total of 28 protein spots (21 genes) were found to have a statistically significant correlation between expression of their protein and mRNA (r>0.2445, p<0.05). This accounts for 17% (28/165) of the 165 protein spots. Among the 69 genes for which only a single protein spot was known (Table 6), 9 genes (9/69, 13%) were observed to show a statistically significant relationship between protein and mRNA abundance (r>0.2445, p<0.05). The proteins whose expression levels were correlated with their mRNA abundance included those involved in signal transduction, carbohydrate metabolism, apoptosis, protein post-translational modification, structural proteins, and heat shock proteins (Table 8).

Individual Isoforms of the Same Protein have Different Protein/mRNA Correlation Coefficients

Of the 165 protein spots, 96 represent protein products of 29 genes with at least two isoforms. Among these 96 protein spots, 19 (19/96 protein spots, 20%) showed a statistically significant correlation between their protein and mRNA expression (r>0.2445, p<0.05) (Table 7), and representing 12 genes (12/29, 41%). Individual isoforms of the same protein demonstrated different protein/mRNA correlation coefficients. For example, 2D PAGE/Western analysis revealed four isoforms of OP 18 differing in regards to isoelectric point but similar in molecular weight. Three of the four isoforms (spots #1492, #1493 and #1494) showed a statistically significant correlation between their protein and mRNA abundance (r=0.3234, 0.3154 and 0.4003 respectively). The forth isoform (spot #1488) showed no correlation between protein and mRNA expression (r=0.0495). Just one of five quantified isoforms of cytokeratin 8 (spot # 439) demonstrated a statistically significant correlation between protein and mRNA abundance (r=0.3049, p<0.05) (Table 7).

In addition to differences in the relationship between mRNA levels and protein expression among separate isoforms, some genes with very comparable mRNA levels showed a 24-fold difference in their protein expression. Genes with comparable protein expression levels also showed up to a 28-fold variance in their mRNA levels.

Lack of Correlation for mRNA and Protein Expression when using Average Tumor Values Across all 165 Protein Spots (98 genes)

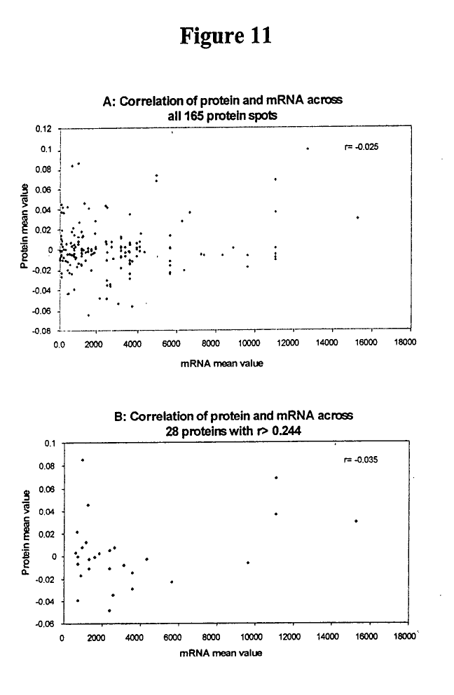

The relationship between mRNA and protein expression was also examined by using the average expression values for all samples. To analyze this relationship using this approach, the average value for each protein or mRNA was generated using all 85 lung tissue samples. The range of normalized average protein values ranged from −0.0646 to 0.0979 (raw value 0.0036 to 4.1947), and the range for mRNA was from 0 to 15260.5 for all 165 individual protein spots. The Spearman correlation coefficient for the whole data set (165 protein spots/98 genes) was −0.025 (FIG. 11A). Even for the 28 protein spots (FIG. 10D) that were found to have a statistically significant correlation between their mRNA and protein, use of the average value resulted in a correlation coefficient value of −0.035 that was not significant (FIG. 11B).

Lack of a Relationship between Protein/mRNA Correlation Coefficients and Average Protein Abundance

To determine if absolute protein level might influence the correlation with mRNA, each protein's mean value (relative abundance) and the Spearman protein/mRNA correlation coefficients among all 85 samples were examined. No relationship between the protein abundance and the correlation coefficients was observed (r=0.039, p>0.05). A detailed analysis of separate subsets of proteins with differing levels of abundance (less than −0.0014, larger than −0.0014, or larger than 0.0077) also showed a lack of correlation between mRNA and protein expression among the 83 (50%), 82 (50%) and 41 (25%) of 165 total protein spots, respectively (r=0.016, 0.08 and 0.172 respectively).

Stage-Related Changes in the Protein/mRNA Correlation Coefficients

To determine whether the 21 genes (28 protein spots) showing a significant correlation between the protein and mRNA expression among all samples demonstrate changes in this relationship during tumor progression, the correlations were examined separately for stage I (n=57) and stage III (n=19) lung adenocarcinomas (Table 8). The number of non-neoplastic lung samples (n=9) was insufficient for a separate correlation analysis of this group. Many of the protein spots represent one of several known protein informs for a given gene. The majority of genes (16/21) did not differ in the protein/mRNA correlation between stage I and stage III tumors indicating a similar regulatory relationship between the mRNA and protein spot. GRP-58, PSMC, SOD1, TPI1 and VIM, however, were found to demonstrate significant differences in the correlation coefficients between stage I and stage III lung adenocarcinomas. For GRP-58, PSMC, and VIM the change in the correlation coefficient was due to a relative increase in protein expression in stage III tumors. For SOD and TPI the change resulted from a relative decrease in expression of this specific protein in stage III tumors.

TABLE 6
Correlation coefficients of protein and mRNA where only one spot was
present on 2D gels

| Spot# | Unigene | Gene name | r* | Protein name |
|---|---|---|---|---|
| 1104 | Hs.184510 | SFN | 0.4337 | 14-3-3 sigma |
| 0994 | Hs.77840 | ANXA4 | 0.4219 | Annexin IV |
| 1314 | Hs.10958 | DJ-1 | 0.3982 | DJ-1 protein/MER5 |
| 1454 | Hs.75428 | SOD1 | 0.3863 | Superoxide dismutase (Cu-Zn) |
| 1638 | Hs.227751 | LGALS1 | 0.3318 | Galectin 1 |
| 0264 | Hs.129548 | HNRPK | 0.3034 | Transformation upregulated nuclear protein |
| 1405 | Hs.111334 | FTL | 0.2849 | Ferritin light chain |
| 0963 | Hs.300711 | ANXA5 | 0.2468 | Annexin V |
| 1252 | Hs.4745 | PSMC | 0.2445 | 26s proteasome p28 |
| 0906 | Hs.234489 | LDHB | 0.4420 | L-lactate dehydrogenase H chain (LDH-B) |
| 1171 | Hs.241515 | COX11 | 0.2310 | COX 11 |
| 1160 | Hs.181013 | PGAM1 | 0.2023 | Phosphoglycerate mutase |
| 0759 | Hs.74635 | DLD | 0.1965 | Dihydrolipoamide dehydrogenase precursor |
| 1193 | Hs.83383 | AOE372 | 0.1932 | Antioxidant enzyme AOE372 |
| 0172 | Hs.3069 | HSPA9B | 0.1872 | GRP75 |
| 0777 | Hs.979 | PDHB | 0.1855 | Pyruvate dehydrogenase E1-beta subunit precursor |
| 1249 | Hs.226795 | GSTP1 | 0.1773 | Glutathione-s-transferase pi (GST-pi) |
| 1685 | Hs.76136 | TXN | 0.1732 | Thioredoxin |
| 1205 | Hs.82314 | HPRT1 | 0.1588 | HG phosphoribosyltransferase |
| 1230 | Hs.279860 | TPT1 | 0.1466 | Translationally controlled tumor protein (TCTP) |
| 0603 | Hs.181357 | LAMR1 | 0.1463 | LAMR |
| 1358 | Hs.28914 | APRT | 0.1399 | Adenine phosphoribosyl transferase |
| 1410 | Hs.82113 | DUT | 0.1213 | dUTP pyrophosphatase (dUTPase) |
| 1825 | Hs.112378 | LIMS1 | 0.1213 | Pinch-2 protein |
| 0871 | Hs.250502 | CA8 | 0.1122 | Carbonic anhydrase-related protein; Syntaxin |
| 0289 | Hs.82916 | CCT6A | 0.1106 | Chaperonin-like protein |
| 1143 | Hs.11465 | GSTTLp28 | 0.0997 | Glutathione-s-transferase homolog (GST homolog) |
| 1456 | Hs.118638 | NME1 | 0.0932 | Nm23 (NDPKA) |
| 1598 | Hs.278503 | RIG | 0.0905 | RIIG (U32331) |
| 1354 | Hs.89761 | ATP5D | 0.0904 | FIFO-type ATP synthase subunit d |
| 1445 | Hs.155485 | HIP2 | 0.0843 | Huntingtin interacting protein 2 (HIP2) |
| 1479 | Hs.177486 | APP | 0.0746 | Amyloid B4A |
| 0608 | Hs.182265 | KRT19 | 0.0439 | Cytokeratin 19 |
| 1071 | Hs.10842 | RAN | 0.0277 | GTP-Binding nuclear protein RAN(TC4) |
| 0991 | Hs.297939 | CTSB | 0.0254 | Cathespin B |
| 0842 | Hs.77274 | PLAU | 0.0248 | Urokinase plasminogen activator |
| 0823 | Hs.198248 | B4GALT1 | 0.0183 | Beta 1,4-galactosyl transferase |
| 0613 | Hs.1247 | APOA4 | 0.0176 | Apolipoprotein A4 (ApoA4) |
| 1338 | Hs.104143 | CLTA | 0.0123 | Clathrin light chain A |
| 0902 | Hs.5123 | SID6-306 | 0.0117 | Cytosolic inorganic pyrophosphatase |
| 1688 | Hs.1473 | GRP | −0.0040 | Preprogastrin-releasing peptide |
| 0265 | Hs.274402 | HSPA1B | −0.0071 | Heat shock induced protein |
| 1414 | Hs.77541 | ARF5 | −0.0096 | ADP-ribosylation factor 1 |
| 0710 | Hs.97206 | HIP1 | −0.0114 | Huntingtin interacting protein 1 (HIP1) |
| 0532 | Hs.170328 | MSN | −0.0132 | Moesin/E |
| 0525 | Hs.284255 | ALPP | −0.0148 | Alkaline phosphate, placental |
| 0513 | Hs.76901 | PDIR | −0.0289 | Protein disulfide isomerase related protein 5 |
| 1659 | Hs.256697 | HINT | −0.0312 | Protein kinase C inhibitor |
| 1262 | Hs.7016 | RAB7 | −0.0362 | Rab 7 protein |
| 0190 | Hs.184411 | ALB | −0.0470 | Albumin |
| 0948 | Hs.2795 | LDHA | −0.0549 | Lactate dehydrogenase-A (LDHA) |
| 0502 | Hs.180532 | GPI | −0.0575 | Hsp89 |
| 0152 | Hs.75410 | HSPA5 | −0.0640 | GRP78 |
| 1054 | Hs.74276 | CLIC1 | −0.0686 | Nuclear chloride channel (RNCC protein) |
| 0709 | Hs.253495 | SFTPD | −0.0936 | Pulmonary surfactant protein D |
| 0867 | Hs.78996 | PCNA | −0.0982 | PCNA |
| 0165 | Hs.180414 | HSPA8 | −0.1014 | Heat shock cognate protein, 71 kDa |
| 1109 | Hs.75103 | YWHAZ | −0.1018 | 14-3-3 zeta/delta |
| 0137 | Hs.554 | SSA2 | −0.1032 | Ro/ss-A antigen |
| 0278 | Hs.4112 | TCP1 | −0.1237 | T-complex protein I, alpha subunit |
| 1769 | Hs.9614 | NPM1 | −0.1738 | B23/numatrin |
| 0089 | Hs.74335 | HSPCB | −0.2049 | Hsp90 |
| 2511 | Hs.153179 | FABP5 | −0.2109 | E-FABP/FABP5 |
| 1739 | Hs.16488 | CALR | −0.2344 | Calreticulin 32 |
| 1138 | Hs.301961 | GSTM4 | −0.2438 | Glutathione-s-transferase M4 (GST m4) |
| 2533 | Hs.77060 | PSMB6 | −0.2512 | Macropain subunit delta |

r*: Correlation coefficient value > 0.2445, p < 0.05.
(Bolded values are significant at p < 0.05)

TABLE 7
Correlation coefficients of protein and mRNA where multiple isoforms were
present on 2D gels

| Spot# | Unigene | Gene name | r* | Protein name |
|---|---|---|---|---|
| 1494 | Hs.81915 | LAP18 | 0.4003 | OP18 (Stathmin) |
| 0957 | Hs.77899 | TPM1 | 0.3930 | Tropomyosins 1-5 |
| 0353 | Hs.289101 | GRP58 | 0.3802 | Protease disulfide isomerase (GRP58) |
| 0855 | Hs.169476 | GAPD | 0.3693 | Glyceraldehyde-3-phosphate dehydrogenase |
| 1198 | Hs.41707 | HSPB3 | 0.3668 | Hsp27 |
| 1203 | Hs.83848 | TPI1 | 0.3395 | Triose phosphate isomerase (TPI) |
| 0523 | Hs.65114 | KRT18 | 0.3335 | Cytokeratin 18 |
| 1492 | Hs.81915 | LAP18 | 0.3234 | OP18 (Stathmin) |
| 1493 | Hs.81915 | LAP18 | 0.3154 | OP18 (Stathmin) |
| 1181 | Hs.78225 | ANXA1 | 0.3102 | Annexin variant I |
| 0439 | Hs.242463 | KRT8 | 0.3049 | Cytokeratin 8 |
| 0505 | Hs.297753 | VIM | 0.2939 | Vimentin |
| 0593 | Hs.297753 | VIM | 0.2809 | Vimentin |
| 1874 | Hs.75313 | AKR1B1 | 0.2790 | Aldose reductase |
| 0935 | Hs.75544 | YWHAH | 0.2775 | 14-3-3 eta |
| 2524 | Hs.78225 | ANXA1 | 0.2612 | Annexin I |
| 2324 | Hs.65114 | KRT18 | 0.2601 | Cytokeratin 18 |
| 1192 | Hs.41707 | HSPB3 | 0.2558 | Hsp27 |
| 0350 | Hs.289101 | GRP58 | 0.2516 | Phospholipase C (GRP58) |
| 0992 | Hs.75313 | AKR1B1 | −0.2460 | Aldose reductase |
| 0861 | Hs.75313 | AKR1B1 | 0.0761 | Aldose reductase |
| 0853 | Hs.75313 | AKR1B1 | −0.0675 | Aldose reductase |
| 2503 | Hs.76392 | ALDH1 | −0.0565 | Aldehyde dehydrogenase |
| 0381 | Hs.76392 | ALDH1 | −0.0371 | Aldehyde dehydrogenase |
| 0371 | Hs.76392 | ALDH1 | −0.0680 | Aldehyde dehydrogenase |
| 1179 | Hs.78225 | ANXA1 | 0.2052 | Annexin variant I |
| 0762 | Hs.78225 | ANXA1 | −0.0739 | Annexin I |
| 0760 | Hs.78225 | ANXA1 | −0.0228 | Annexin I |
| 2506 | Hs.217493 | ANXA2 | 0.2223 | Lipocotin (annexin II) |
| 0772 | Hs.217493 | ANXA2 | 0.2080 | Lipocotin (annexin II) |
| 0723 | Hs.217493 | ANXA2 | 0.0701 | Lipocotin |
| 1239 | Hs.93194 | APOA1 | 0.1133 | Apolipoprotein A1 (ApoA1) |
| 1237 | Hs.93194 | APOA1 | −0.0373 | Apolipoprotein A1 (ApoA1) |
| 1234 | Hs.93194 | APOA1 | −0.0894 | Apolipoprotein A1 (ApoA1) |
| 0428 | Hs.25 | ATP5B | 0.0080 | ATP synthase beta subunit precursor |
| 0427 | Hs.25 | ATP5B | 0.0122 | ATP synthase beta subunit precursor |
| 0424 | Hs.25 | ATP5B | −0.0992 | ATP synthase beta subunit precursor |
| 0863 | Hs.75106 | CLU | −0.0483 | Apolipoprotein J (ApoJ) |
| 0780 | Hs.75106 | CLU | −0.0443 | Apolipoprotein J (ApoJ) |
| 1527 | Hs.119140 | EIF5A | −0.0726 | eIF-5A |
| 1484 | Hs.119140 | EIF5A | −0.0376 | eIF-5A |
| 1728 | Hs.5241 | FABP1 | −0.1916 | L-FABP |
| 1712 | Hs.5241 | FABP1 | −0.0473 | L-FABP |
| 0947 | Hs.169476 | GAPD | 0.1745 | Glyceraldehyde-3-phosphate dehydrogenase |
| 1232 | Hs.75207 | GLO1 | 0.2249 | Glyoxalase-I |
| 1229 | Hs.75207 | GLO1 | 0.0450 | Glyoxalase-1 |
| 1595 | Hs.158300 | HAP1 | −0.0137 | huntingtin-associated protein 1 (neuroan 1) |
| 1810 | Hs.75990 | HP | −0.4672 | Alpha-haptoglobin |
| 1459 | Hs.75990 | HP | 0.0802 | Alpha-haptoglobin |
| 1458 | Hs.75990 | HP | −0.0305 | Alpha haptoglobin |
| 0619 | Hs.75990 | HP | 0.0461 | B-haptoglobin |
| 0615 | Hs.75990 | HP | −0.0034 | B-haptoglobin |
| 1250 | Hs.41707 | HSPB3 | −0.1024 | Hsp27 |
| 0549 | Hs.79037 | HSPD1 | 0.1074 | Hsp60 |
| 0338 | Hs.79037 | HSPD1 | 0.2265 | Hsp60 |
| 0333 | Hs.79037 | HSPD1 | 0.1383 | Hsp60 |
| 0331 | Hs.79037 | HSPD1 | 0.1603 | Hsp60 |
| 2381 | Hs.65114 | KRT18 | 0.2016 | Cytokeratin 18 |
| 0535 | Hs.65114 | KRT18 | 0.1106 | Cytokeratin 18 |
| 0529 | Hs.65114 | KRT18 | 0.1279 | Cytokeratin 18 |
| 0528 | Hs.65114 | KRT18 | 0.0414 | Cytokeratin 18 |
| 0527 | Hs.65114 | KRT18 | 0.0436 | Cytokeratin 18 |
| 0514 | Hs.65114 | KRT18 | 0.0733 | Cytokeratin 18 |
| 0451 | Hs.242463 | KRT8 | −0.0111 | Cytokeratin 8 |
| 0446 | Hs.242463 | KRT8 | 0.0347 | Cytokeratin 8 |
| 0444 | Hs.242463 | KRT8 | −0.1311 | Cytokeratin 8 |
| 0443 | Hs.242463 | KRT8 | 0.0942 | Cytokeratin 8 |
| 1488 | Hs.81915 | LAP18 | 0.0495 | OP18 (Stathmin) |
| 0321 | Hs.75655 | P4HB | −0.0546 | PDI (proly-4-OH-B) |
| 0320 | Hs.75655 | P4HB | −0.0041 | PDI (proly-4-OH-B) |
| 1063 | Hs.75323 | PHB | 0.0441 | Prohibitin |
| 0837 | Hs.75323 | PHB | 0.1402 | Prohibitin |
| 0326 | Hs.297681 | SERPINA1 | −0.0227 | Alpha-1-antitripsin |
| 0322 | Hs.297681 | SERPINA1 | −0.0277 | Alpha-1-antitripsin |
| 0241 | Hs.297681 | SERPINA1 | −0.0148 | Alpha-1-antitripsin |
| 1280 | Hs.301254 | SFTPA1 | −0.1488 | Pulmonary surfactant-associated protein |
| 1278 | Hs.301254 | SFTPA1 | −0.2040 | Pulmonary surfactant-associated protein |
| 0866 | Hs.73980 | TNNT1 | 0.1162 | Troponin T |
| 0778 | Hs.73980 | TNNT1 | 0.0740 | Troponin T |
| 1213 | Hs.83848 | TPI1 | 0.0024 | Triose phosphate isomerase (TPI) |
| 1210 | Hs.83848 | TPI1 | 0.0490 | Triose phosphate isomerase (TPI) |
| 1207 | Hs.83848 | TPI1 | −0.1615 | Triose phosphate isomerase (TPI) |
| 1204 | Hs.83848 | TPI1 | 0.0209 | Triose phosphate isomerase (TPI) |
| 1202 | Hs.83848 | TPI1 | 0.0721 | Triose phosphate isomerase (TPI) |
| 1161 | Hs.83848 | TPI1 | 0.2265 | Triose phosphate isomerase (TPI) |
| 1052 | Hs.77899 | TPM1 | −0.1040 | Tropomysin clean-product |
| 1039 | Hs.77899 | TPM1 | −0.2999 | Cytoskeletal tropomyosin |
| 1035 | Hs.77899 | TPM1 | −0.3821 | Tropomyosin |
| 0783 | Hs.77899 | TPM1 | 0.0757 | Tropomyosins 1-5 |
| 1574 | Hs.194366 | TTR | −0.0065 | Transthyretin |
| 0809 | Hs.194366 | TTR | 0.0399 | Transthyretin multimere |
| 2202 | Hs.76118 | UCHL1 | −0.0220 | Ubiquitin carboxyl-terminal hydrolase isozyme L1 |
| 1246 | Hs.76118 | UCHL1 | −0.1261 | Ubiquitin carboxyl-terminal hydrolase isozyme L1 |
| 1242 | Hs.76118 | UCHL1 | 0.1473 | Ubiquitin carboxyl-terminal hydrolase isozyme L1 |
| 0606 | Hs.297753 | VIM | 0.0951 | Vimentin |
| 0594 | Hs.297753 | VIM | −0.2664 | Vimentin derived protein (vid4) |
| 0508 | Hs.297753 | VIM | 0.1008 | Vimentin derived protein (vid2) |
| 0419 | Hs.297753 | VIM | 0.0032 | Vimentin derived protein (vid1) |
| 1279 | Hs.75544 | YWHAH | 0.0059 | 14-3-3 eta |

r*: Correlation coefficient value > 0.2445, p < 0.05.
(Bolded values are significant at p < 0.05)

TABLE 8
Stage-dependent analysis of protein-mRNA correlation coefficients

| Spot# | Gene name | r (stage I) | r (stage III) | Function |
|---|---|---|---|---|
| 1874 | AKR1B1 | 0.269 | 0.106 | Carbohydrate metabolism; electron transporter |
| 2524 | ANXA1 | 0.184 | 0.572 | Phospholipase inhibitor; signal transduction |
| 0994 | ANXA4 | 0.660 | 0.362 | Phospholipase inhibitor |
| 0963 | ANXA5 | 0.241 | 0.390 | Phospholipase inhibitor; calcium binding; phospholipid binding |
| 1314 | DJ-1 | 0.363 | 0.354 | Signal transduction |
| 1405 | FTL | 0.126 | 0.358 | Iron storage protein |
| 0855 | GAPD | 0.243 | 0.581 | Carbohydrate metabolism (glycolysis regulation) |
| 0350 | GRP58 | 0.327 | −0.087 | Signal transduction; protein disulfide isomerase |
| 0264 | HNRPK | 0.360 | 0.243 | RNA-binding protein (RNA processing/modification) |
| 1192 | HSPB3 | 0.457 | 0.633 | Heat shock protein |
| 0523 | KRT18 | 0.115 | 0.371 | Structural protein |
| 0439 | KRT8 | 0.323 | 0.436 | Structural protein |
| 1492 | LAP18 | 0.483 | 0.663 | Signal transduction; cell growth and maintenance |
| 1638 | LGALS1 | 0.200 | 0.528 | Apoptosis; cell adhesion; cell size control |
| 1252 | PSMC | 0.253 | 0.060 | Protein degradation |
| 1104 | SFN | 0.465 | 0.475 | Signal transduction (protein kinase C inhibitor) |
| 1454 | SOD1 | 0.352 | 0.079 | Oxidoreductase |
| 1203 | TPI1 | 0.378 | 0.009 | Carbohydrate metabolism |
| 0957 | TPM1 | 0.475 | 0.225 | Structural protein (muscle); control of heart |
| 0593 | VIM | −0.054 | 0.556 | Structural protein |
| 0935 | YWHAH | 0.283 | 0.210 | Signal transduction |

r: correlation coefficient
Bolded values indicate a significant different between stage I and stage III.

### Example 4

Proteomic Analysis of Cytokeratin Isoforms Uncovers Association with Survival in Lung Adenocarcinoma

A. Materials and Methods

Sample Acquisition and Preparation

Sequential patients seen between May 1991 and July 2000 by General Thoracic Surgery at the University of Michigan Hospital for resection of stage I and III lung adenocarcinoma were evaluated for inclusion in this study. Consent was received from all patients and the project approved by Institutional Review Board. Patient medical records were reviewed and patient identifiers coded to protect confidentiality. Tumor tissues and adjacent non-neoplastic lung tissues were acquired immediately after resection and carried to the laboratory in Dulbecco's modified Eagle medium (Life Technologies, Gaithersburg Pa.) on ice. This included 64 stage I, 29 stage III lung adenocarcinomas, as well as 10 non-neoplastic lung samples (Table 9). A portion of each tumor and/or lung tissue in were embedded OCT (Miles Scientific, Naperville, Ill.), and frozen in isopentane cooled with liquid nitrogen for cryostat sectioning, and then stored at −80° C. Hematoxylin- and eosin-stained cryostat sections (5 m), prepared from tumor pieces to be utilized for mRNA isolation, were evaluated by a study pathologist, and compared to H&E sections made from paraffin blocks of the same tumor. Specimens were excluded based on unclear or mixed histology (e.g., adenosquamous), tumor cellularity less than 70%, potential metastatic origin as indicated by previous tumor history, extensive lymphocytic infiltration, extensive fibrosis, prior chemotherapy or radiotherapy. Tumors were histopathologically divided into two categories: bronchial-derived, if they exhibited invasive features with architectural destruction, and bronchioloalveolar, if they exhibited preservation of the lung architecture.

2-D Page

Protein samples were solubilized in standard lysis buffer (9.5 M urea, 20 μL non-ionic detergent (Nonidet P-40), 20 μL of ampholines (pH 3.5-10; Pharmacia/LKB, Piscataway, N.J.), 20 μL of 2-mercaptoethanol, and 0.89M phenylmethylsulfonyl fluoride per milliliter of deionized water). Sample volumes of between 15-30 μL were immediately applied to isoelectric focusing gels containing 50 μL ampholytes per milliliter (pH 3.5-10). First dimensional separation was performed using 700 V for 16 hours, and then 1000 V for 2 hours at room temperature. Second dimensional separation was accomplished using an 18 cm×18 cm gel containing an acrylamide gradient of 11.4-14 g/dl. Samples were run in 20 gel batches. After separation, the protein spots were visualized by a photochemical silver-based staining technique.

Detection and Quantification

Following two-dimensional separation, each gel was scanned using a Kodak CCD camera. A 1024×1024 pixel format was used yielding pixel widths of 163 μm where each pixel had 256 possible gray-scale values (optical density). Spot detection was accomplished by Bio Image Visage System software (Bioimage Corp., Ann Arbor, Mich.). Each gel generated 1600-2200 detectable spots, with 820 well-defined spots in most samples, chosen to obtain quantitative measurements. The integrated intensity, which is the value of each spot, was calculated as the measured optical density units×square millimeters. Then spots from each gel were matched to the 820 spots on a “master” gel (Kuick et al., Electrophoresis 12:736 [1991]) to allow for the identification of identical polypeptides between each gel. A total of 250 spots were chosen as ubiquitously expressed reference spots to allow adjustment for variations in protein loading and gel staining. Each of the 820 spots was then mathematically adjusted in relation to the reference spots (Kuick et al., Electrophoresis 8:199 [1987]).

Mass Spectrometry

Protein spots identified for analysis were extracted from preparative 2-D gels of extracts from A549 lung adenocarcinoma cell lysates (obtained from ATCC) or lung adenocarcinoma tissue lysates (obtained from patient). The conditions were identical to the analytical 2-D gel, except there was a 30% greater protein loading, and were silver-stained by successive incubations in 0.02% sodium thiosulfate for 2 min, 0.1% silver nitrate for 40 min, and then 0.014% formaldehyde plus 2% sodium carbonate for 10 min. Identification of proteins was performed by trypsin digestion followed by either matrix-assisted laser desorption ionization time-of-flight mass spectrometry (MALDI-TOF MS) in the Perseptive Voyager Biospectrometry Workstation (PerSeptive Biosystem, Framingham, Mass., USA), or nanoflow capillary liquid chromatography coupled with electrospray tandem mass spectrometry (ESI MS/MS) in a Q-TOF micro (Micromass, Manchester, UK). MALDI-TOF MS gave a “fingerprint” for each spot based on the molecular weight of trypsin-digested products and compared the resulted mass with known trypsin digest databases using the MSFit database searching. MS/MS spectra produced by ESI MS/MS were automatically processed and searched against a non-redundant database using ProteinLynx Global SERVER.

RNA Isolation for Microarrays

RNA was isolated from 75 of the tumors and the 10 normal samples used in the protein studies. Two contiguous, 2 mm³ samples were removed for RNA and protein isolation respectively. Total cellular RNA was isolated using Trisol reagent (Life Technologies, Gaithersburg, Pa.) and subjected to further purification using RNeasy columns (Qiagen Inc., Valencia, Calif.). Five micrograms of total RNA was used as template. All protocols used for mRNA reverse transciptase, second strand synthesis, production of cDNA and RNA amplification, hybridization, and washing conditions for the 6800 gene HUGeneFL oligonucleotide arrays are as provided by the manufacturer (Affymetrix, Santa Clara, Calif.). More detailed information is provided in Giordano, et al. (Am. J. Pathol. 159:1231-1238 [2001]).

Mutational Analysis of K-ras

Genomic DNA was isolated from each tumor sample and fifty ng was subjected to PCR amplification using the primers that encompass codons 12 and 13 of the K-ras gene. The sequences of forward and reverse primers are 5′ TATAAGGCCTGCTGAAAAT 3′ (SEQ ID NO:31) and 5′ CCTGCACCAGTAATATGC 3′ (SEQ ID NO:32), respectively. Two ng of purified PCR products containing the exon 1 of the K-ras gene were then subjected to thermal cycle sequencing with an internal nested primer (5′ AGGCCTGCTGAAAATGACT 3′; SEQ ID NO:33) and resolved in 8% urea PAGE gels, dried, and exposed to Phosphor-Image screens and visualized using a Phosphor-Inage scanner (Molecular Dynamics, Sunnyvale, Calif.). The mutations were determined by comparing each tumor DNA sequences of K-ras 12th and 13th codon to its wild type sequence GGTGGC (SEQ ID NO:34).

Immunohistochemical Staining

Diagnosis for all primary lung adenocarcinomas used in this study were confirmed by a board-certified pathologist. A tissue microarray block was assembled based on the best morphological areas of the tumors used in this study according to the method of Kononen (Nat Med. 4:844 [1998]). Deparaffinized sections of the pulmonary adenocarcinoma tissue microarray were microwave pretreated in citric acid to retrieve antigenicity. The sections were incubated with 1% hydrogen peroxide for 60 minutes to inhibit endogenous peroxidase activity at room temperature. Following blocking to reduce nonspecific binding, the sections were incubated with primary antibodies overnight at 4° C. Antibodies included anti-p53 (M-7001;1:500), anti-CK19 (M0772;1:500), and anti-CK7 (OV-TL 12/30;1:500) from DAKO Corporation, Carpenteria, Calif.; anti-CK7 (K72.7; 1:500, Neomarkers, Fremont, Calif.); anti-CK18 (DC-10;1:500), and anti-CK8 (TS 1; 1:500) from Novo Castra Laboratories, Newcastle, UK. The immuno-complex was visualized by the immunoglobulin enzyme bridge technique using a Vector ABC-peroxidase kit (Vector Laboratories, Burlingame, Calif.). The enzyme substrate was 3,3′ diaminobenzidine tetrachloride, resulting in a brown reactant. The sections were lightly counter-stained with hematoxylin.

2D Western Blotting

Protein extracts of A549 lung adenocarcinoma cells were run on 2D gels using the identical conditions as used for the analytical 2D gels. The separated proteins were transferred onto polyvinylidene fluoride membranes and incubated for 2 h at room temperature with a blocking buffer consisting of TBST (Tris-buffered saline, 0.01% Tween 20) and 5% nonfat dry milk. Individual membranes were washed and incubated with monoclonal antibodies listed above for immunohistochemistry and used at 1:500 dilution. After additional washes with TBST, the membranes were incubated with a secondary antibody conjugated with horseradish peroxidase (HRP) at a 1:5000 dilution for 1 h, further washed and then incubated 1 min with ECL (enhanced chemiluminescence) (Pierce, Ill.).

Statistical Analysis

A student's T-test was used for comparing protein expression in lung adenocarcinoma vs. uninvolved (normal) lung. An F-test was used to compare between values of other clinical-pathological variables by constructing background-adjusted protein spot size quantification. The transform x→log (1+x) was applied to all protein expression measurements. A probability (p) level <0.05 (two-sided) was considered statistically significant. Potential associations between mRNA and protein expression were made by calculating Spearman correlation coefficients for individual genes and gene products within the same samples.

Survival time was defined as the interval in months between the day of operation for the lung adenocarcinoma and the date of lung-cancer related death or last follow-up. A total of 682 protein spots were included in the survival analysis after the elimination of spots that were absent from at least 60% of all the gels. Univariate Cox proportional hazards regression methods were used to identify proteins associated with variability in survival times.

B. Results

Cytokeratin Isoforms Show Altered Expression in Lung Adenocarcinoma

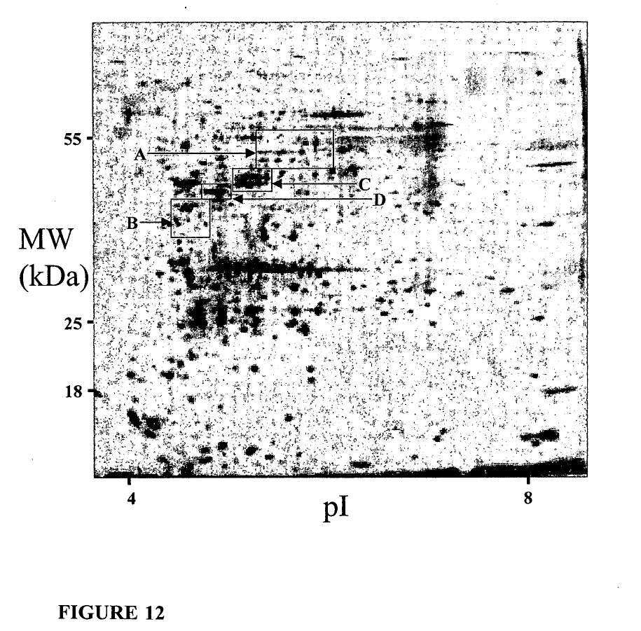

Systematic identification of proteins detected in lung adenocarcinoma tumor and cell lines have uncovered to date over 300 differentially expressed proteins expressed in our lung cancer protein database (Oh et al., Proteomics 1:1303 [2001]). Four cytokeratin types (CK7, CK8, CK18 and CK19) have been identified (FIG. 12), each of which demonstrated multiple protein isoforms in tumor samples (Table 10). All four cytokeratins had at least one or more isoforms that showed significant increases (p<0.05) in lung adenocarcinomas compared to normal lung. For CK19, two isoforms were over-expressed and one isoform (#608) was under-expressed in lung adenocarcinoma.

Cytokeratin Isoforms Associated with Patient Survival and Other Clinical-Pathologic Variables

Univariate Cox proportional hazards regression analysis revealed that one CK8, one CK19, and all five CK7 isoforms identified by MS were significantly associated (p<0.05) with patient survival. Interestingly, the five CK7 isoforms had estimated molecular weights ranging from 36.7 kDa to 41.7 kDa, and estimated pI's ranging from 4.6 to 4.9 that are quite different from the reported native CK7 molecular weight of 51.3 kDa and pI of 5.5. The estimated pI and molecular weights for CK 8, 18, and 19 isoforms were relatively similar to their reported standards (Moll et al., Cell 31:11 [1982]).

Of the 21 cytokeratin isoforms examined, only a subset showed a significant correlation to other clinical-pathological variables (Table 11). F-tests showed that some CK isoforms such as CK7 (#691), which had a relationship to survival, was decreased in bronchial-derived tumors and those with a positive lymphocytic response. None of the other CK7 isoforms showed a relationship to any clinical-pathological variables. However three CK8 isoforms showed relationships to differentiation, positive lymphocytic response, or K-ras mutation status. One CK18 isoform was also increased in tumors with a positive lymphocytic response, and of the three CK19 isoforms, one was increased in tumors with p53 and K-ras mutations, and one was decreased in poorly differentiated tumors.

2D Western Blot Analysis of Cytokeratins

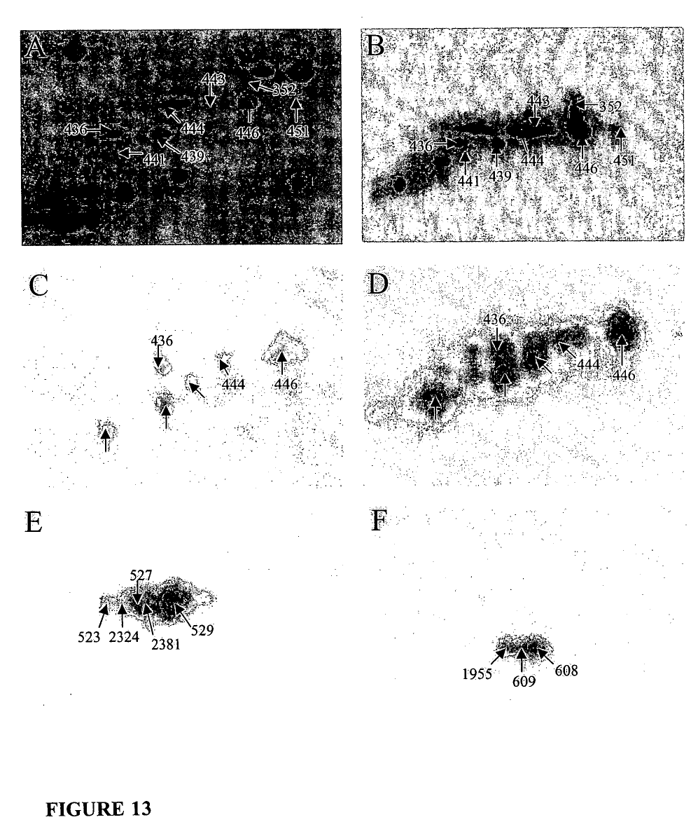

The patterns of reactivity of cytokeratins with 2D Western blot analysis (FIG. 13) of the regions of 2D gels demonstrating the immunoreactivity shown in FIG. 1 (Boxes A-D) was determined. Outlined box B indicated the region from which the five over-expressed CK7 proteins identified by MS were located. The CK8 antibody identified at least 15 separate immunoreactive proteins (FIG. 13B) including the eight isoforms that were identified by MS (FIG. 12, Box A), and for which quantitative analysis was performed (FIG. 13A). Two different monoclonal antibodies to CK7 representing two different clones were examined (FIGS. 13C, 13D). Both antibodies detected some isoforms in common as well as distinct CK7 isoforms. The location of the immunoreactive CK7 isoforms are very close to the location to the CK8 fragments (FIG. 13B) and three of the isoforms (436, 444, 446) appear to overlap between CK7 and CK8 fragments. All other fragments are clearly distinct from each other, although their location is similar. The redundancy of the immunostaining may be the result of the extensive sequence similarity of CK7 and CK8 (Glass et al., J Cell Biol 107:1337 [1988]; Leube et al., Differentiation 33:69-85 [1986]).

The immunoreactive CK7 proteins are the same pI and MW predicted from their sequence, however, neither of the two CK7 antibodies directed against distinct clones reacted with the 5 isoforms identified by MS suggesting that these smaller CK7 proteins represented cleavage products that lacked the epitopes recognized by these monoclonal antibodies. Quantitative values for the immunoreactive CK7 isoforms could not be obtained from the silver-stained gels due to low abundance, although these forms are relatively abundant in the A549 lung cell line. All five CK7 isoforms were significantly associated with survival (p<0.05), significantly increased in the adenocarcinomas and frequently expressed (58.1% vs. 39.8%) relative to normal lung (Table 10).

Five CK18 and three CK19 isoforms were detected using MS and 2D Westerns (FIG. 13E, F) and their protein abundance quantitatively analyzed (Table 10). Unlike CK7 and CK8, all CK18 and CK19 isoforms demonstrated similar MW but varied in pI. Interestingly, all five CK18 isoforms were significantly increased in the adenocarcinomas and detected frequently in these tumors relative to normal lung (15-60%), but an association with survival was not observed. The isoforms #529 and #523 are the most abundant in both normal lung and lung adenocarcinomas. All three CK19 isoforms were significantly increased in the adenocarcinoma but only one isoform (#1955), the most positively charged isoform, was associated with survival and demonstrated the greatest differential expression between normal lung and the adenocarcinomas. This isoform was significantly associated with the CK19 mRNA levels suggesting that the level of transcription in part can influence its abundance.

Cytokeratin mRNA levels and Correlation to Other Genes

Because the same lung adenocarcinomas and non-neoplastic lung samples that were quantitatively analyzed for protein expression were examined at the level of mRNA abundance using oligonucleotide arrays, correlation coefficients between these cytokeratin proteins and the levels of their corresponding mRNAs could be determined.

A positive correlation was observed between the CK protein expression values for two CK7 isoforms (#165, #2091) and CK7 mRNA levels (Table 4). One CK8 isoform (#439), two CK18 isoforms (#523, #2324) and one CK19 isoform (#1955) were also significantly associated with mRNA levels of corresponding genes. Importantly, univariate Cox proportional hazards regression analysis revealed that CK7, CK8, CK18 and CK19 mRNA showed a significant relationship to survival and all four were significantly increased at the mRNA level in the adenocarcinomas relative to normal lung (Table 13). Further, correlation analysis showed that the mRNA levels for all four CK genes were significantly correlated (p<0.05) to each other (Table 14), suggesting these increases may reflect a common mechanism. Further analysis of 4966 expressed genes examined in these samples revealed two genes encoding liver-specific bHLH-Zip transcription factor and smooth and non-muscle myosin light chain polypeptide to be significantly correlated to all four CKs.

Cellular Localization of Candidate Proteins

Utilizing the same antibodies used for 2D Western blot analysis (FIG. 13), the expression of the four CK forms was confirmed as present specifically in lung adenocarcinoma by immunohistochemistry of tumor tissue arrays. These arrays contained the same tumors examined in this study at both the protein and mRNA levels. A very similar pattern and cytoplasmic localization of all four of these CK proteins was observed in the lung adenocarcinomas with much lower levels detected in normal lung. Interestingly, all four CK showed relatively abundant expression in the epithelial counterpart of the fetal lung, consistent with previous studies (Broers et al., Differentiation 40:119 [1989]).

TABLE 9
Clinical-pathological characteristics of 93 lung
adenocarcinoma patients
Age

| | |
|---|---|
| <65 | 49 |
| >65 | 44 |

Gender

| | |
|---|---|
| Female | 53 |
| Male | 40 |

Race

| | |
|---|---|
| White | 76 |
| Non-White (Asian, | 8 |
| Black) | |
| unknown | 9 |

Smoking

| | |
|---|---|
| Non Smoking | 10 |
| Smoking | 79 |
| Unknown | 4 |

Stage

| | |
|---|---|
| I | 64 |
| III | 29 |

T Status

| | |
|---|---|
| T1 | 49 |
| T2-4 | 44 |

N Status

| | |
|---|---|
| N0 | 68 |
| N1-2 | 25 |

Classification

| | |
|---|---|
| Bronchoalveolar | 14 |
| Bronch-Derived | 76 |
| Mixed | 3 |

Differentiation

| | |
|---|---|
| Well | 22 |
| Moderate | 47 |
| Poor | 23 |

Lymphocytic response

| | |
|---|---|
| Yes | 41 |
| No | 52 |

Angiolymphatic invasion

| | |
|---|---|
| Yes | 16 |
| No | 77 |

Tumor Location

| | |
|---|---|
| Left lobe | 31 |
| Right lobe | 62 |

p53 nuclear accumulation

| | |
|---|---|
| Positive | 19 |
| Negative | 69 |

K-ras mutational status

| | |
|---|---|
| Positive | 34 |
| Negative | 40 |

TABLE 10
Cytokeratin expression in lung adenocarcinoma and their frequency of
expression in tumor and normal lung samples

| | Protein | est. | est. | Univariate Cox | | Normal (n = 10) | Tumor (n = 93) | | Fold change | Tumor | |
|---|---|---|---|---|---|---|---|---|---|---|---|
| Spot # | name | MW | pl | Model P-value | Coefficient Beta | mean ± SD | mean ± SD | P (T-test) | (tumor/normal) | frequency** | fre |
| 691 | CK 7 | 41.7 | 4.9 | 0.025 | 0.406 | 0.049 ± 0.066 | 0.231 ± 0.182 | <0.0001 | 4.714 | 58.1% | |
| 871 | CK 7 | 41.7 | 4.7 | 0.005 | −0.659 | 0.125 ± 0.167 | 0.159 ± 0.177 | 0.5566 | — | — | |
| 1968 | CK 7 | 41.0 | 4.6 | 0.014 | 0.516 | 0.303 ± 0.104 | 0.493 ± 0.282 | 0.0002 | 1.627 | 39.80% | |
| 2091 | CK 7 | 40.4 | 4.6 | 0.029 | 0.356 | 0.325 ± 0.355 | 0.246 ± 0.211 | 0.3227 | — | — | |
| 2165 | CK 7 | 36.7 | 4.7 | 0.019 | −0.487 | 0.136 ± 0.049 | 0.119 ± 0.099 | 0.3845 | — | — | |
| 852 | CK 8 | 54.3 | 5.6 | 0.442 | — | 0.050 ± 0.023 | 0.070 ± 0.081 | 0.0768 | — | — | |
| 436 | CK 8 | 50.6 | 5.2 | 0.101 | — | 0.313 ± 0.168 | 0.237 ± 0.123 | 0.1939 | — | — | |
| 439 | CK 8 | 50.7 | 5.4 | 0.033 | 0.382 | 0.165 ± 0.080 | 0.343 ± 0.132 | <0.0001 | 2.078 | 49.5% | |
| 441 | CK 8 | 49.7 | 5.2 | 0.681 | — | 0.048 ± 0.062 | 0.103 ± 0.057 | 0.0219 | 2.158 | 10.8% | |
| 443 | CK 8 | 53.1 | 5.5 | 0.481 | — | 0.029 ± 0.060 | 0.042 ± 0.066 | 0.5254 | — | — | |
| 444 | CK 8 | 52.7 | 5.4 | 0.594 | — | 0.008 ± 0.026 | 0.039 ± 0.058 | 0.0062 | 4.796 | 23.7% | |
| 446 | CK 8 | 53.1 | 5.6 | 0.990 | — | 0.019 ± 0.030 | 0.103 ± 0.103 | <0.0001 | 5.453 | 50.5% | |
| 451 | CK 8 | 54.1 | 5.7 | 0.814 | — | 0.013 ± 0.028 | 0.027 ± 0.052 | 0.1783 | — | — | |
| 523 | CK 18 | 45.4 | 5.1 | 0.393 | — | 0.126 ± 0.074 | 0.304 ± 0.191 | <0.0001 | 2.413 | 50.5% | |
| 527 | CK 18 | 45.3 | 5.2 | 0.221 | — | 0.044 ± 0.060 | 0.103 ± 0.102 | 0.0186 | 2.331 | 15.1% | |
| 529 | CK 18 | 46.0 | 5.4 | 0.108 | — | 0.121 ± 0.097 | 0.388 ± 0.191 | <0.0001 | 3.219 | 60.2% | |
| 2324 | CK 18 | 46.0 | 5.2 | 0.642 | — | 0.000 ± 0.000 | 0.117 ± 0.168 | <0.0001 | N/A | 36.6% | |
| 2381 | CK 18 | 46.0 | 5.3 | 0.084 | — | 0.000 ± 0.000 | 0.124 ± 0.111 | <0.0001 | N/A | 49.5% | |
| 609 | CK 19 | 43.6 | 4.7 | 0.724 | — | 0.057 ± 0.081 | 0.132 ± 0.133 | 0.0215 | 2.305 | 26.9% | |
| 1955 | CK 19 | 43.6 | 4.6 | 0.026 | 0.433 | 0.010 ± 0.032 | 0.163 ± 0.142 | <0.0001 | 16.171 | 67.7% | |
| 608 | CK 19 | 43.7 | 4.8 | 0.567 | — | 0.987 ± 0.512 | 0.512 ± 0.348 | 0.0170 | 0.519 | 50.5% | |

*Normal avg + 2SD was used as cut-off value for proteins that were overexpressed in tumor samples. Normal avg − 2SD was used as cut-off value for proteins that were underexpressed in tumor samples
**Frequency of expression in tumor samples is percent of samples greater than cut-off value.
***Frequency of expression in normal samples is percent of samples greater than cut-off value.
—is placed where value is insignificant due to univariate cox model p-value > 0.05 or t-test p-value > 0.05
N/A represents a fold change that cannot be expressed due to normal mean value being zero.

TABLE 11
Relationship between cytokeratins and different clinical-pathological variables

| | | | Expression in | Expression in | | | Expression in |
|---|---|---|---|---|---|---|---|
| | | Expression | bronchial-derived | poorly-differentiated | | | samples with |
| | | in stage III | samples | samples (vs. | Expression | Expression | presence of |
| | Protein | (vs. stage I) | bronchioloalveolar) | (vs. well, moderate) | in p53 + (vs. p53 −) | in K-ras + (vs. K-ras −) | lymphocytic response |
| Spot # | name | (n = 29 vs. 64) | (n = 73 vs. 14) | (n = 23 vs. 22, 47) | (n = 19 vs. 69) | (n = 34 vs. 40) | (n = 39 vs. 51) |
| 691 | CK 7 | | down (p = 0.021) | | | | down (p = 0.011) |
| 352 | CK 8 | | | up (p = 0.027) | | | |
| 436 | CK 8 | up (p = 0.013) | | | | | down (p = 0.012) |
| 451 | CK 8 | | | | | down (p = 0.038) | |
| 2381 | CK 18 | | | | | | up (p = 0.032) |
| 608 | CK 19 | | | | up (p = 0.027) | up (p = 0.028) | |
| 609 | CK 19 | | | down (p = 0.003) | | | |

Other CK spots that did not significantly change in expression: 871, 1968, 2091, 2165 (CK7); 439, 441, 443, 444, 446 (CK8); 527, 529, 2324 (CK18); 1955 (CK19).

TABLE 12
Correlation coefficients between cytokeratin
protein and mRNA values

| Spot # | Gene Name | Correlation Coefficient (r)* |
|---|---|---|
| 2165 | CK7 | 0.35 |
| 2091 | CK7 | 0.29 |
| 1968 | CK7 | 0.14 |
| 691 | CK7 | 0.10 |
| 871 | CK7 | −0.18 |
| 439 | CK8 | 0.30 |
| 441 | CK8 | 0.18 |
| 436 | CK8 | 0.11 |
| 443 | CK8 | 0.09 |
| 352 | CK8 | 0.04 |
| 446 | CK8 | 0.03 |
| 451 | CK8 | −0.01 |
| 444 | CK8 | −0.13 |
| 523 | CK18 | 0.33 |
| 2324 | CK18 | 0.26 |
| 2381 | CK18 | 0.20 |
| 529 | CK18 | 0.13 |
| 527 | CK18 | 0.04 |
| 1955 | CK19 | 0.39 |
| 609 | CK19 | 0.15 |
| 608 | CK19 | 0.04 |

*p < 0.05 if r > 0.25

TABLE 13
Cytokeratin mRNA expression

| | | | Normal | Tumor | Fold change | |
|---|---|---|---|---|---|---|
| Gene | P (Cox | Beta | Mean | Mean | (tumor/ | P (T- |
| Name | model) | Coefficient | (n = 10) | (n = 86) | normal) | test) |
| CK7 | 0.0004 | 0.0003 | 951 | 2146 | 2.26 | <0.0001 |
| CK8 | 0.0934 | 0.0001 | 1368 | 3436 | 2.51 | <0.0001 |
| CK18 | 0.0006 | 0.0003 | 1310 | 3826 | 2.92 | <0.0001 |
| CK19 | 0.0009 | 0.0003 | 952 | 2522 | 2.65 | <0.0001 |

TABLE 14
Correlation coefficients between cytokeratins and other mRNA

Correlation Coefficient (r)*

| Gene Name | CK7 | CK8 | CK18 | CK19 |
|---|---|---|---|---|
| CK7 | 1.00 | 0.53 | 0.62 | 0.43 |
| CK8 | | 1.00 | 0.75 | 0.52 |
| CK18 | | | 1.00 | 0.40 |
| CK19 | | | | 1.00 |
| LISCH7 | 0.41 | 0.52 | 0.43 | 0.40 |
| MYL6 | 0.43 | 0.61 | 0.41 | 0.44 |

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for characterizing lung tissue in a subject, comprising:
a) providing a lung tissue sample; and
b) detecting a decreased or increased expression relative to a non-cancerous lung tissue control of a marker selected from the group consisting of AOE372, ATP5D, B4GALT, Ppase, GRP58, GSTM4, P4HB, TPI, and UCHL1, thereby characterizing said lung tissue sample.

2. The method of claim 1, wherein said expression is increased at least 4.0 fold relative to said non-cancerous lung tissue control.

3. The method of claim 1, wherein said detecting the presence of expression of said marker comprises detecting the presence of mRNA corresponding to said marker.

4. The method of claim 1, wherein said detecting the presence of expression of said marker comprises detecting the presence of a polypeptide corresponding to said marker.

5. The method of claim 1, wherein said characterizing said lung tissue comprises identifying a stage of lung cancer in said lung tissue.

6. The method of claim 5, wherein said stage is selected from the group consisting of stage I lung cancer, stage II lung cancer, and stage III lung cancer.

7. The method of claim 1, further comprising the step of c) providing a prognosis to said subject.

8. A method of characterizing lung cancer, comprising
a) providing a lung cancer tissue sample; and
b) detecting the level of expression relative to a non-cancerous lung tissue control of a marker selected from the group consisting of GRP-58, PSMC, VIM, SOD, TPI, thereby characterizing said lung cancer tissue sample.

9. The method of claim 8, wherein an increased level of GRP-58, PSMC, and VIM is indicative of stage III lung cancer.

10. The method of claim 8, wherein a decreased level of SOD and TPI is indicative of stage III lung cancer.

11. A method of predicting survival in lung cancer patients, comprising
a) providing lung cancer tissue sample from a subject;
b) detecting the presence or absence of a marker selected from the group consisting of CK19, CK7, and CK8 in said lung cancer tissue sample.

12. The method of claim 11, wherein the presence of said marker is indicative of decreased survival of said subject.

13. A lung cancer expression profile map comprising gene expression level information for two or more markers selected from the group consisting of BAG 1, CASP4, FADD, P63, 5T4, ITGA2, KRT18, KRT19, KRT7, LAMB1, TMSB4X, TUBA1, BMP2, CDC6, H2AFZ, PDAP1, POLD3, REG1A, S100P, SERPINE1, STX1A, ADM, AKAP12, ARHE, DEFB1, GRB7, INHA, ITK, NACA, STC1, TNFAIP6, VEGF, VLDLR, WNT1, WNT10B, HSPA8, ERBB2, FXYD3, HLA-B, HPCAL1, P2RX5, PEX7, SLC20A1, SLC2A1, VDAC2, ALDH8, ALDOA, ATP2B1, CDS1, CSTB, CTSL, CYP24, FUCA1, FUT3, GAPD, GCNT1, HMBS, KYNU, MLN64, MSH3, MT2A, NME2, NP, PACE, PDE7A, PLGL, PPIF, PTPRCAP, RPC, SC4MOL, SLC1A6, UBC, UGP2, UQCRC2, COPEB, CRK, DBP, GARS, HRB, HSU53209, PRDM2, RELA, RPS26, RPS3, RPS6 KB1, SUI1, TIEG, TMF1, B1, FEZ2, HPIP, KLAA0005, KIAA0020, KIAA0084, KLAA0153, KIAA0263, KIAA0317 and MGB1.

14. The lung cancer expression profile map of claim 13, wherein said map is digital information stored in computer memory.
